(19) United States
(12) Patent Application Publication
(10) Pub. No.: US 2008/0188680 A1
(43) Pub. Date: Aug. 7, 2008

(54) Process Improvement Using Solubility Characteristics

(76) Inventor: Michael J. Gentilcore, Maryland Heights, MO (US)

Correspondence Address: Anthony R. Kinney;Mallinckrodt Inc., 675 McDonnell Blvd., Hazelwood, MO, 63042, US

(21) Appl. No.: 11/817,263

(22) PCT Filed: Feb. 21, 2006
(86) PCT No.: PCT/US06/06135

§ 371 Date: Aug. 28, 2007

Related U.S. Application Data
(60) Provisional application No. 60/657,012, filed on Feb. 28, 2005.

(51) Int. Cl. C07C 205/04; C07C 63/04

(52) U.S. Cl. 562/434; 562/493

(57) ABSTRACT

The present invention is directed to a process of maximizing the solubility of a nonelectrolyte solute in a solvent by operating within an optimal temperature range at conditions wherein the nonelectrolyte solute is not a pure liquid. In particular, the process of the present invention is directed to conversion of a carboxylic acid compound to an ester under conditions wherein the solubility of the ester in an alcoholic solvent approaches ideal solubility behavior.

### BACKGROUND OF THE INVENTION

The present invention generally relates to processes having an operating temperature range at which solubility of a non-electrolyte solute in a solvent is maximized. For example, this process is advantageous in increasing conversion of a solid reactant to a non-electrolyte solute by operating at or slightly above a temperature wherein the solubility of the solute in the solvent is increasing rapidly. In one exemplary embodiment, the process can be used to increase conversion of carboxylic acids to high boiling esters in an alcohol solvent in the presence of a solid acid catalyst.

Many chemical processes depend on the solubility of solids in liquid solvents to define key process steps. Such processes include crystallization to isolate product in good yield, recrystallization to remove impurities, filtration where a dissolved product solution is filtered from a solid catalyst, and reactions where a solid reactant must be dissolved to participate in a chemical conversion.

One such reaction is the esterification of a carboxylic acid in an alcohol in the presence of an acid catalyst. Various processes for preparing esters using a carboxylic acid substrate, alcohol and acid catalyst have been proposed. In U.S. Pat. No. 6,002,041, a homogeneous acid catalyst is used (e.g., sulfuric acid). Use of a homogeneous acid catalyst introduces the complication of purification of the ester product. The product ester cannot be efficiently dried in the presence of sulfuric acid and, thus, an additional crystallization step is necessary. The additional step adds to the processing time and capital costs for solids isolation and handling equipment. In U.S. Pat. No. 5,426,199 a heterogeneous acid catalyst specifically designed to reduce the production of an ether byproduct is used (e.g. a strongly acidic cation-exchange resin). The strongly acidic cation-exchange resin is made up of crosslinked, vinylaromatic polymer beads; the beads are surface functionalized with strongly acidic functional groups to a cation-exchange capacity of from about 0.1 to about 2.5 meq/g and have an inner volume of unfunctionalized polymer. The surface functionalized polymer beads favor the formation of esters over ethers.

There is a need for processes for improving chemical conversion of a solid reactant to a non-electrolyte solute in a solvent, such as the chemical conversion of carboxylic acids to product esters in the presence of a solid acid catalyst. It has been found that the process of the invention can be applied to such processes to improve the economic feasibility of a particular process step by improving reaction kinetics or payloads, or reducing energy costs, solvent costs and waste disposal costs.

### SUMMARY OF THE INVENTION

Among the various aspects of the present invention is a process having an operating temperature range at which the solubility of a non-electrolyte solute in a solvent is maximized. The process comprises forming a mixture of the non-electrolyte solute and solvent at or slightly above a temperature wherein the solubility of the solute is increasing rapidly. More specifically, the present invention is directed to a process for increasing conversion of a solid reactant to a non-electrolyte solute by operating at or slightly above a temperature wherein the solubility of the solute is increasing rapidly. For example, esters can be formed from carboxylic acid substrates and alcohol in greater yield by such a process as compared to commercial processes.

Another aspect of the present invention is a process having an operating temperature range, Tₘᵢₙ to Tₘₐₓ, at which solubility of a non-electrolyte solute in a solvent is maximized, the process comprising forming a mixture comprising the non-electrolyte solute and the solvent, wherein the solubility of the solute in the solvent is less than the ideal solubility at temperature, T, the temperature of the mixture being between Tₘᵢₙ and Tₘₐₓ. The activity coefficient of the solute in a two component mixture of the solute in the solvent at a temperature, T, γₛₒₗᵤₜₑ, is derived from equation (5);

$$ln {γ}_{solute} {x}_{solute} = \frac{- Δ {H}_{solute}^{F}}{R} ( \frac{1}{T} - \frac{1}{{T}_{m ( solute )}} ) . \qquad ( 5 )$$

Variables are defined as follows: xₛₒₗᵤₜₑ is the mole fraction of dissolved solute in a saturated two component mixture of solute and solvent at a temperature, T, in K; R is the ideal gas constant in cal/K·mol; −ΔH^{F}ₛₒₗᵤₜₑ is the heat of fusion in cal/g·mol of the solute; Tₘ₍ₛₒₗᵤₜₑ₎ is the absolute melting temperature of the solute, in K; Tₘᵢₙ is a temperature, in K, where an absolute value of γₛₒₗᵤₜₑ is greater than one and less than about 10 and the second derivative of a function of γₛₒₗᵤₜₑ with respect to temperature becomes a positive value; and Tₘₐₓ is a temperature, in K, which is greater than Tₘᵢₙ and less than (Tₘᵢₙ+0.8(Tₘ₍ₛₒₗᵤₜₑ₎−Tₘᵢₙ)).

Yet another aspect of the present invention is a process having an operating temperature range, Tₘᵢₙ to Tₘₐₓ, at which solubility of a non-electrolyte solute in a solvent is maximized, the process comprising forming a mixture comprising the non-electrolyte solute and the solvent, wherein the solubility of the solute in the solvent is less than the ideal solubility at a temperature, T, the temperature of the mixture being between Tₘᵢₙ and Tₘₐₓ. Variables are defined as follows: xₛₒₗᵤₜₑ is the mole fraction of dissolved solute in a saturated two component mixture of solute and solvent at a temperature, T; Tₘ₍ₛₒₗᵤₜₑ₎ is the normal melting temperature of the solute; Tₘᵢₙ is a temperature where the second derivative of a function of xₛₒₗᵤₜₑ with respect to temperature becomes a negative value; and Tₘₐₓ is a temperature greater than Tₘᵢₙ and less than (Tₘᵢₙ+0.8(Tₘ₍ₛₒₗᵤₜₑ₎−Tₘᵢₙ)).

Still another aspect of the present invention is a process having an operating temperature range, Tₘᵢₙ to Tₘₐₓ, at which solubility of a non-electrolyte solute in a solvent is maximized, the process comprising forming a mixture comprising the non-electrolyte solute and the solvent, wherein the solubility of the solute in the solvent is less than the ideal solubility at a temperature, T, the temperature of the mixture being between Tₘᵢₙ and Tₘₐₓ. The variables are defined as follows: wt. %ₛₒₗᵤₜₑ is the wt. % of dissolved solute in a saturated two component mixture of solute and solvent at a temperature, T; Tₘ₍ₛₒₗᵤₜₑ₎ is the normal melting temperature of the solute; Tₘᵢₙ is a temperature where the second derivative of a function of wt. %ₛₒₗᵤₜₑ with respect to temperature changes becomes a negative value; and Tₘₐₓ is a temperature greater than Tₘᵢₙ and less than about (Tₘᵢₙ+0.8(Tₘ₍ₛₒₗᵤₜₑ₎−Tₘᵢₙ)).

A further aspect of the present invention is a process for preparing an ester from a carboxylic acid comprising (a) reacting a carboxylic acid substrate with an alcohol to form a reaction mixture comprising the carboxylic acid substrate, the alcohol, and the ester wherein at least about 25% of the carboxylic acid substrate measured on a molar basis is converted to the ester; and (b) contacting the reaction mixture with a solid acid catalyst to form a reaction product comprising the ester wherein at least about 97% of the carboxylic acid substrate measured on a molar basis is converted to ester.

Yet a further aspect of the present invention is a process for separating a mixture of an alcohol, an ether and water, the process comprising introducing an aqueous mixture of an alcohol and an ether into a distillation column; separating the mixture to produce water and a vapor comprising the ether and the alcohol; introducing the vapor exiting an upper portion of the column into a lower portion of a knockback condenser; and condensing the vapor in the knockback condenser to form an ether-rich vapor exiting an upper portion of the knockback condenser and an alcohol-rich liquid exiting the lower portion of the knockback condenser, wherein the ether-rich vapor contains at least about 50 wt. % of the ether in the mixture.

### BRIEF DESCRIPTION OF THE DRAWINGS

FIG. 1 is a solubility curve for a saturated solution of dimethyl-5-nitro-isophthalate (referred to as “diester”) in methanol.

FIG. 2 is a graph of the activity coefficient for a saturated solution of diester in methanol with respect to the temperature; the second derivative of the function of the activity coefficient with respect to the temperature is also graphed.

FIG. 3 is a graph of the mole fraction for a saturated solution of diester in methanol with respect to the temperature; the second derivative of the function of the mole fraction with respect to the temperature is also graphed.

FIG. 4 is a graph of the weight percent of a saturated solution of diester in methanol with respect to the temperature; the second derivative of the function of the weight percent with respect to the temperature is also graphed.

FIG. 5 is a schematic diagram of an exemplary process for conversion of 5-nitro-isophthalic acid to dimethyl-5-nitro-isophthalate.

FIG. 6 is a schematic diagram of a knockback condenser.

FIG. 7 is a schematic diagram of a heat and material balance flow sheet simulation of a partial condenser and the streams, which enter and exit the condenser used for Example 7.

FIG. 8 is a schematic diagram of a heat and material balance flow sheet simulation of a knockback condenser and the streams, which enter and exit the condenser used for Example 7.

### DETAILED DESCRIPTION OF THE PREFERRED EMBODIMENTS

Among the various aspects of the present invention is a process having an operating temperature range at which the solubility of a non-electrolyte solute product in a solvent is maximized. It has been discovered that processes involving non-ideal solutions can be improved by operating within such a temperature range. For example, conversion of a solid reactant to a non-electrolyte solute product in a solvent can be increased by conducting the reaction within this temperature range wherein the solubility of the solute is increasing rapidly. This process is particularly advantageous for non-ideal solutions at a temperature wherein (i) the activity coefficient of the solute is greater than one and less than about 10 and the second derivative of the function of the activity coefficient with respect to temperature is a positive value; (ii) the second derivative of the function of the weight percentage of solute with respect to temperature is a negative value; or (iii) the second derivative of the function of the mole fraction of the solute with respect to temperature is a negative value.

In order to determine the temperature range at which the solubility of a non-electrolyte solute product in a solvent is maximized, the process of the invention can be applied.

A. Application of Solubility Theory to Non-Ideal Solutions

The ideal solubility is dependent on the operating temperature, melting point of the solute, and heat of fusion of the solute at the normal melting point. The ideal solubility is calculated as a mole fraction from equation (1)

$$ln ( {x}_{solute ideal} ) = \frac{- Δ {H}_{solute}^{F}}{R} ( \frac{1}{T} - \frac{1}{{T}_{m ( solute )}} ) . \qquad ( 1 )$$

Solubility data can be compared to the ideal solubility. Two component solubility data (solute and solvent) at various temperatures can be easily calculated as a mole fraction of the solute in solution. The ratio of the mole fraction ideal solubility, x_{solute ideal} (calculated at the same temperature as the solubility data point) to each mole fraction solubility data point, x_{solute actual} is the activity coefficient, γₛₒₗᵤₜₑ. These relationships are expressed mathematically, per equation (2)

γₛₒₗᵤₜₑ=x_{solute ideal}/x_{solute actual} (2).

Equation (2) can be substituted into equation (1) to give equation (3) below.

$$ln {γ}_{solute} {x}_{solute actual} = \frac{- Δ {H}_{solute}^{F}}{R} ( \frac{1}{T} - \frac{1}{{T}_{m ( solute )}} ) \qquad ( 3 )$$

In order to solve equation (3) for the activity coefficient, three experimental values are necessary: (i) the melting point of the solute, (ii) the heat of fusion of the solute in cal/g·mol and (iii) at least one value for the solubility of the specific solute in a specific solvent. Assuming the heat of fusion of the solute is constant over the range of temperatures, the relationship in equation (3) allows the mathematical transformation of the temperature and solubility data into an activity coefficient for the solute at each temperature. The terms in equation (3) are defined as follows: γₛₒₗᵤₜₑ is the activity coefficient of the solute, xₛₒₗᵤₜₑ is the mole fraction of the solute at a given temperature, T, in K; −ΔH^{F}ₛₒₗᵤₜₑ is the heat of fusion in cal/g·mol of the solute, R is the ideal gas constant in cal/K·mol, T is the temperature of a given value for xₛₒₗᵤₜₑ in K; and Tₘ₍ₛₒₗᵤₜₑ₎ is the melting temperature of the solute in K.

Further, this comparison of actual to ideal solubility is particularly advantageous for solutes that show solubility behavior that is not ideal at and below the normal boiling point of the solvent. Particularly, this comparison to select an optimum process temperature is particularly advantageous for solutes wherein the activity coefficient of the solute at the normal boiling point of the solvent is greater than about 4. The magnitude of the activity coefficient at a given temperature indicates how closely the solubility of the solute in the solvent correlates to the ideal solubility of the solute calculated by equation (1) at that given temperature. An activity coefficient of one shows ideal behavior and an increasing value of the activity coefficient shows solubility that is departing from ideal solubility. The magnitude of activity coefficients at different temperatures shows how the solubility of the solute varies with temperature. Typically, as the temperature increases the solubility of the solute in the solvent becomes closer to the ideal solubility.

This procedure for selecting operating conditions that optimize solute solubility while minimizing operating temperature is applicable to solutes that are non-electrolytes. Stated another way, equation (3) is used for molecules that do not dissociate or ionize in solution. Additionally, equation (3) applies to pure solids. Solubility data at one or more given temperature values may be collected by methods known in the art. A solubility data set contains one value for xₛₒₗᵤₜₑ and a corresponding temperature, wherein the xₛₒₗᵤₜₑ value corresponds to the mole fraction of the solute contained in a saturated two component system of the solute in the solvent. One method for determining the solubility of a solute in a solvent is a gravimetric method as described in Example 1. Solubility data sets for more than one temperature provide added confidence in Van Laar parameters produced from a linear regression of equation (4). One or more solubility data sets for the solute may be taken at convenient temperatures below the atmospheric boiling point of the solvent.

After calculation of activity coefficients using equation (3), preferably, if more than one solubility data set was measured, the activity coefficients and the corresponding temperature data can be fitted by linear regression to a form of the van Laar expression, equation (4), wherein Aₛₒₗᵤₜₑ and Bₛₒₗᵥₑₙₜ are derived.

$$\frac{1}{{( RT ln {γ}_{solute} )}^{0.5}} = \frac{{( {A}_{solute} )}^{0.5} {x}_{solute}}{{B}_{solvent} {x}_{solvent}} + \frac{1}{{( {A}_{solute} )}^{0.5}} \qquad ( 4 )$$

Aₛₒₗᵤₜₑ and Bₛₒₗᵥₑₙₜ are correlation parameters and the values are specific to the solute and solvent components, xₛₒₗᵥₑₙₜ is the mole fraction of the solvent in a two component mixture; and other parameters are as defined herein above. Equation (4) is in the form of Y=mX+b. Upon plotting Y versus X, wherein Y is 1/(RTlnγₛₒₗᵤₜₑ)^{0.5} and X is xₛₒₗᵤₜₑ/xₛₒₗᵥₑₙₜ, the intercept of the line, b, is 1/(Aₛₒₗᵤₜₑ)^{0.5} and the slope of the line, m, equals (Aₛₒₗᵤₜₑ)^{0.5}/Bₛₒₗᵥₑₙₜ. See Gentilcore, M. J., Chemical Engineering Progress 2004, 100(3), 38.

Alternatively, when one solubility data set is used, the values for Aₛₒₗᵤₜₑ and Bₛₒₗᵥₑₙₜ are obtained from equations (5), (6) and (7). In this case, the quotient Aₛₒₗᵤₜₑ/Bₛₒₗᵥₑₙₜ is derived from equation (5)

$$\frac{{A}_{solute}}{{B}_{solvent}} = \frac{{V}_{solute}^{L}}{{V}_{solvent}^{L}} \qquad ( 5 )$$

wherein Aₛₒₗᵤₜₑ and Bₛₒₗᵥₑₙₜ are defined above, Vₛₒₗᵤₜₑ^{L} is the quotient of the molecular weight in g/mol of the solute divided by the density in g/cm³ of the pure liquid melt of the solute and Vₛₒₗᵥₑₙₜ^{L} is the quotient of the molecular weight in g/mol of the solvent divided by the density in g/cm³ of the solvent. Equation (5) is most reliable for non-electrolytes that are non-polar solutes and solvents. Once the quotient Aₛₒₗᵤₜₑ/Bₛₒₗᵥₑₙₜ is calculated from equation (5), equation (6) is solved for Aₛₒₗᵤₜₑ wherein xₛₒₗᵤₜₑ is the value from the solubility data set, T is the temperature in K at which xₛₒₗᵤₜₑ was measured, xₛₒₗᵥₑₙₜ is calculated from equation (2) and γₛₒₗᵤₜₑ is the value calculated from equation (3). Bₛₒₗᵥₑₙₜ is determined from equation (7) using the quotient Aₛₒₗᵤₜₑ/Bₛₒₗᵥₑₙₜ determined from equation (5) and Aₛₒₗᵤₜₑ determined from equation (6).

$$\begin{matrix}RT ln {γ}_{solute} = {{A}_{solute} ( 1 + \frac{{A}_{solute}}{{B}_{solvent}} \frac{{x}_{solute}}{{x}_{solvent}} )}^{- 2} & ( 6 ) \\ {B}_{solvent} = {A}_{solute} / ( {A}_{solute} / {B}_{solvent} ) & ( 7 )\end{matrix}$$

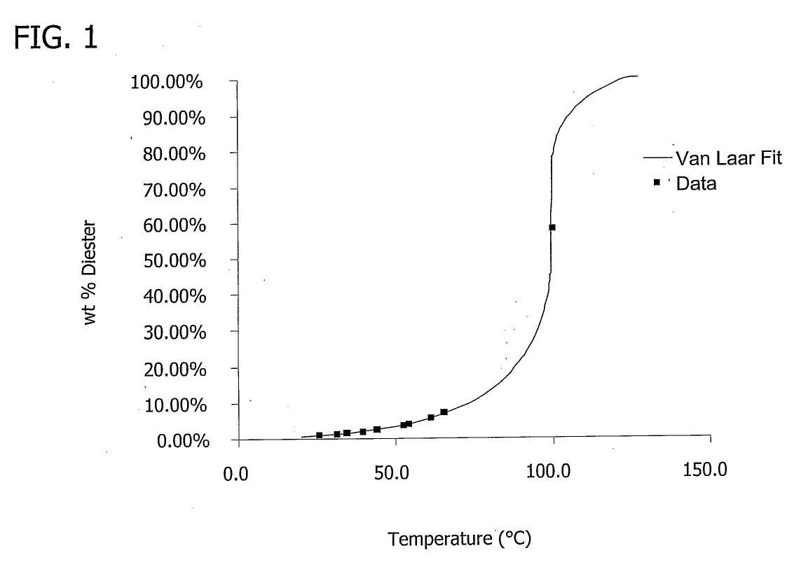

Once Aₛₒₗᵤₜₑ and Bₛₒₗᵥₑₙₜ are calculated from either method, the value of xₛₒₗᵤₜₑ and xₛₒₗᵥₑₙₜ for a given temperature can be calculated by a trial and error routine from equations (6), (3) and the relationship xₛₒₗᵤₜₑ=1−xₛₒₗᵥₑₙₜ. This methodology is exemplified in Example 1 and graphically presented in FIG. 1.

If the optimum process temperature determined above is greater than the atmospheric boiling point of the solvent, the reaction vessel may be pressurized to reach the optimum process temperature.

Alternatively, a temperature range, Tₘᵢₙ to Tₘₐₓ, can be determined at which solubility of a non-electrolyte solute in a solvent is maximized. A mixture comprising the non-electrolyte solute and the solvent is formed, wherein the activity coefficient, γₛₒₗᵤₜₑ, of the solute in the solvent is less than about 4, and the temperature of the mixture is between Tₘᵢₙ and Tₘₐₓ. The activity coefficient γₛₒₗᵤₜₑ of the solute in a two component mixture of the solute in the solvent at a temperature, T, is derived from equation (3); Tₘᵢₙ is a temperature, in K, where an absolute value of γₛₒₗᵤₜₑ is greater than one and less than about 10 and the second derivative of a function of γₛₒₗᵤₜₑ with respect to temperature becomes a positive value; and Tₘₐₓ is a temperature, in K, which is greater than Tₘᵢₙ and less than (Tₘᵢₙ+0.8(Tₘ₍ₛₒₗᵤₜₑ₎−Tₘᵢₙ)). In other embodiments, the process conditions may vary depending on the process system and Tₘₐₓ is greater than Tₘᵢₙ and less than about (Tₘᵢₙ+0.7(Tₘ₍ₛₒₗᵤₜₑ₎−Tₘᵢₙ)), (Tₘᵢₙ+0.6(Tₘ₍ₛₒₗᵤₜₑ₎−Tₘᵢₙ)), (Tₘᵢₙ+0.5(Tₘ₍ₛₒₗᵤₜₑ₎−Tₘᵢₙ)), (Tₘᵢₙ+0.4(Tₘ₍ₛₒₗᵤₜₑ₎−Tₘᵢₙ)), (Tₘᵢₙ+0.3(Tₘ₍ₛₒₗᵤₜₑ₎−Tₘᵢₙ)), or (Tₘᵢₙ+0.2(Tₘ₍ₛₒₗᵤₜₑ₎−Tₘᵢₙ)).

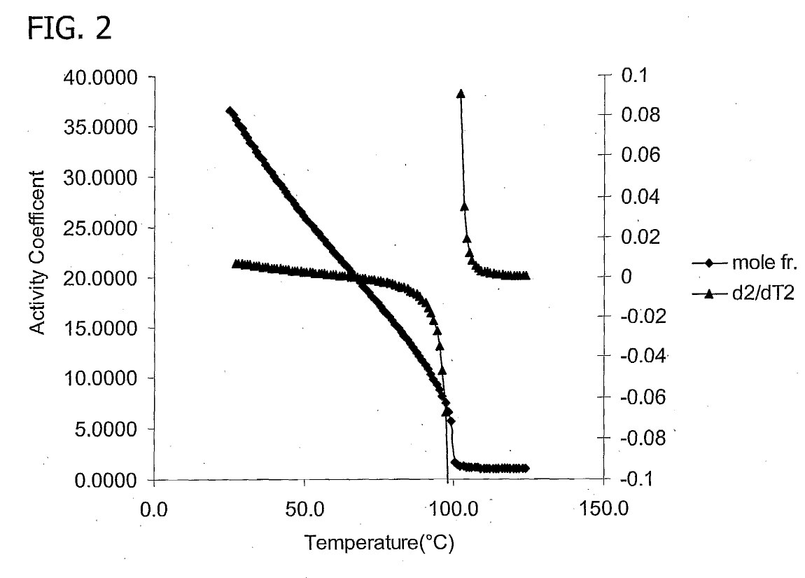

As an exemplary embodiment, solubility data for a non-electrolyte solute product is graphically presented wherein the activity coefficient, γₛₒₗᵤₜₑ, of the solute in the solvent is along the y-axis and temperature is along the x-axis of a two dimensional plot. In addition, the second derivative of the function of the activity coefficient, γₛₒₗᵤₜₑ, of the solute in the solvent with respect to temperature is plotted. For a non-ideal solution, a rapid change in γₛₒₗᵤₜₑ of the solute will be observed at a particular temperature below the melting point of the solute. This rapid change in γₛₒₗᵤₜₑ with temperature corresponds to a change in the value of the second derivative. The temperature wherein the second derivative is zero corresponds to the temperature at which the function of γₛₒₗᵤₜₑ with respect to temperature is at an inflection point. At the inflection point, the solubility of the solute in the solvent is rapidly increasing and continues increasing until the temperature of the two-component mixture reaches the melting point of the solute. This relationship of the solubility of the solute with respect to temperature also holds for functions of the wt. %ₛₒₗᵤₜₑ and xₛₒₗᵤₜₑ with respect to temperature as described below. As an example, solubility data for the diester of Example 1 was used to prepare a graph (FIG. 2) of the activity coefficient, γₛₒₗᵤₜₑ, of diester in methanol with respect to temperature. In addition, the second derivative of the function of the activity coefficient, γₛₒₗᵤₜₑ, of diester in methanol with respect to temperature is plotted. A rapid change in γₛₒₗᵤₜₑ of the diester is observed at about 100° C., and the second derivative of the function of the activity coefficient, γₛₒₗᵤₜₑ, of the diester in methanol with respect to temperature changes from zero to a positive value at about 100° C.

In yet another embodiment, the process of the present invention is a process having an operating temperature range, Tₘᵢₙ to Tₘₐₓ, at which solubility of a non-electrolyte solute product in a solvent is maximized. A mixture comprising a non-electrolyte solute and a solvent is formed, wherein the solubility of the solute in the solvent is less than the ideal solubility at temperature, T, and the temperature of the mixture is between Tₘᵢₙ and Tₘₐₓ; Tₘᵢₙ is a temperature where the second derivative of a function of xₛₒₗᵤₜₑ with respect to temperature becomes a negative value and Tₘₐₓ is greater than Tₘᵢₙ and less than (Tₘᵢₙ+0.8(Tₘ₍ₛₒₗᵤₜₑ₎−Tₘᵢₙ)). In other embodiments, the process conditions may vary depending on the process system and Tₘₐₓ is greater than Tₘᵢₙ and less than about (Tₘᵢₙ+0.7(Tₘ₍ₛₒₗᵤₜₑ₎−Tₘᵢₙ)), (Tₘᵢₙ+0.6(Tₘ₍ₛₒₗᵤₜₑ₎−Tₘᵢₙ)), (Tₘᵢₙ+0.5(Tₘ₍ₛₒₗᵤₜₑ₎−Tₘᵢₙ)), (Tₘᵢₙ+0.4(Tₘ₍ₛₒₗᵤₜₑ₎−Tₘᵢₙ)), (Tₘᵢₙ+0.3(Tₘ₍ₛₒₗᵤₜₑ₎−Tₘᵢₙ)), or (Tₘᵢₙ+0.2(Tₘ₍ₛₒₗᵤₜₑ₎−Tₘᵢₙ)).

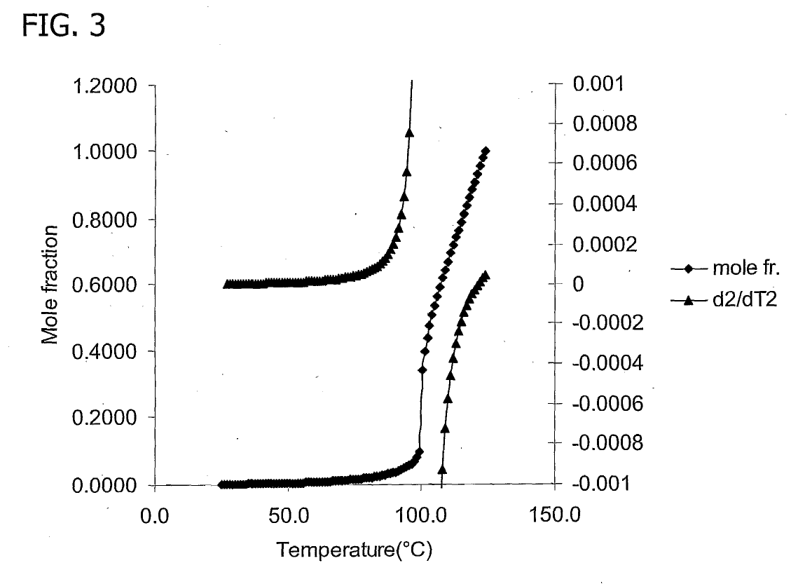

As with the plot of the activity coefficient described above, a graph of the mole fraction, xₛₒₗᵤₜₑ, of the solute in a solvent with respect to temperature as well as the second derivative thereof will reveal an optimal operating temperature at which a rapid increase in mole fraction is observed and the second derivative becomes a negative value. In an exemplary embodiment, solubility data for the diester of Example 1 was used to prepare a graph (FIG. 3) of the mole fraction, xₛₒₗᵤₜₑ, of diester in methanol with respect to temperature. In addition, the second derivative of the function of the mole fraction, xₛₒₗᵤₜₑ, of diester in methanol with respect to temperature is plotted. This plot also shows a rapid change in solubility of the diester at about 100° C.

In another embodiment, the process of the present invention is a process having an operating temperature range, Tₘᵢₙ to Tₘₐₓ, at which solubility of a non-electrolyte solute product in a solvent is maximized. A mixture comprising a non-electrolyte solute and a solvent is formed, wherein the solubility of the solute in the solvent is less than the ideal solubility at temperature, T, and the temperature of the mixture is between Tₘᵢₙ and Tₘₐₓ; Tₘᵢₙ is a temperature where the second derivative of a function of wt. %ₛₒₗᵤₜₑ with respect to temperature becomes a negative value and Tₘₐₓ is greater than Tₘᵢₙ and less than (Tₘᵢₙ+0.8(Tₘ₍ₛₒₗᵤₜₑ₎−Tₘᵢₙ)). In other embodiments, the process conditions may vary depending on the process system and Tₘₐₓ is greater than Tₘᵢₙ and less than about (Tₘᵢₙ+0.7(Tₘ₍ₛₒₗᵤₜₑ₎−Tₘᵢₙ)), (Tₘᵢₙ+0.6(Tₘ₍ₛₒₗᵤₜₑ₎−Tₘᵢₙ)), (Tₘᵢₙ+0.5(Tₘ₍ₛₒₗᵤₜₑ₎−Tₘᵢₙ)), (Tₘᵢₙ+0.4(Tₘ₍ₛₒₗᵤₜₑ₎−Tₘᵢₙ)), (Tₘᵢₙ+0.3(Tₘ₍ₛₒₗᵤₜₑ₎−Tₘᵢₙ)), or (Tₘᵢₙ+0.2(Tₘ₍ₛₒₗᵤₜₑ₎−Tₘᵢₙ)).

As with the plot of the activity coefficient described above, a graph of the weight percentage, wt. %ₛₒₗᵤₜₑ, of the solute in a solvent with respect to temperature as well as the second derivative thereof will reveal an optimal operating temperature at which a rapid increase in weight percentage of solute is observed and the second derivative becomes a negative value.

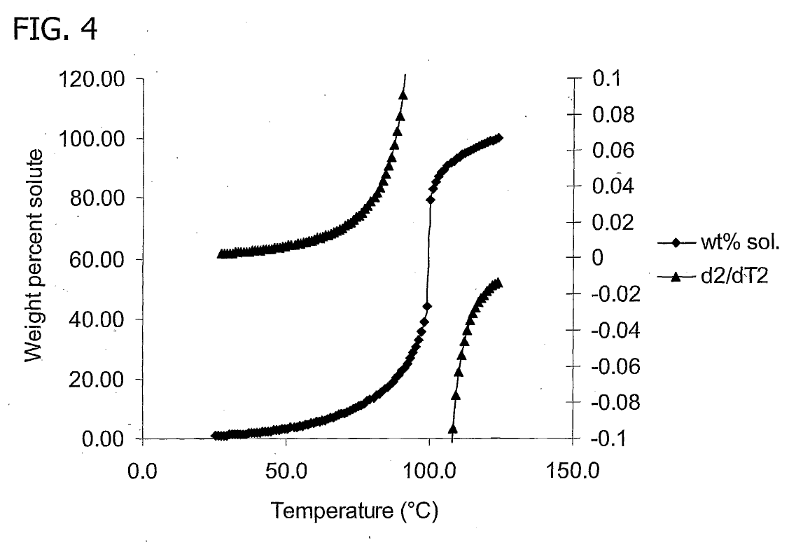

In an exemplary embodiment, solubility data for diester was used to prepare a graph (FIG. 4) of the weight percent, wt. %ₛₒₗᵤₜₑ, of diester in methanol with respect to temperature. In addition, the second derivative of the function of the wt. %ₛₒₗᵤₜₑ, of diester in methanol with respect to temperature is plotted. This plot also shows a rapid change in solubility of diester at about 100° C.

In a presently preferred embodiment, the solid reactant is a carboxylic acid substrate, the non-electrolyte solute is an ester, and the solvent is an alcohol. The solubility of the ester in the alcohol is measured at multiple temperatures to plot the solubility curve and determine the temperature at which the solubility of the ester increases rapidly to determine the minimum operating temperature for the reaction.

B. Process for Chemical Conversion of a Solid Reactant to a Non-Electrolyte Solute in a Solvent

By generation of a solubility curve for a non-electrolyte solute product in a solvent as described above, the information obtained can be used to optimize process conditions that are difficult to explore in the laboratory (e.g., elevated pressures and temperatures). The higher solubility of the solute at elevated temperatures may be advantageous for several reasons. For example, higher solubility would allow for higher payloads for a reaction where the product is kept in solution and filtered away from other solids such as heterogeneous catalysts, solid absorbents, or byproduct salts. Another advantage of higher solubility is faster reaction kinetics, especially for reactions where solids are predominantly out of solution at lower temperatures. Further, higher solubility is advantageous if it allows reduction in the volume of solvent; it can reduce energy costs, solvent costs and waste disposal costs.

In one embodiment, the process of the present invention is described using an esterification reaction as an example. An esterification reaction is a reaction where a carboxylic acid substrate reacts with an alcohol in a reaction mixture to produce a corresponding product ester, i.e. the non-electrolyte solute product, and water as illustrated in Reaction Scheme A wherein RCOOH is a carboxylic acid substrate, R′OH is an alcohol and RCOOR′ is a product ester.

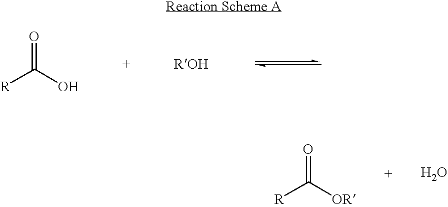

As depicted, the carboxylic acid substrate and alcohol are part of an equilibrium reaction with the product ester and water; stated another way, this reaction proceeds measurably in both the forward and reverse directions.

Another embodiment of the present invention is an etherification reaction illustrated in Reaction Scheme B

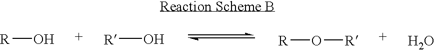

wherein two alcohol substrate molecules (ROH and R′OH) combine to form a product ether (ROR′) and water. The two alcohol substrate molecules can be the same or different, thus producing symmetrical or asymmetrical ether products. Again, as with the esterification reaction, the alcohol substrates are part of an equilibrium reaction with the product ether and water. In addition, the process of the invention could be applied to a transetherification reaction analogous to the transesterification reaction of Reaction Scheme C.

In yet another embodiment, the process of the present invention is directed to a transesterification reaction illustrated in Reaction Scheme C

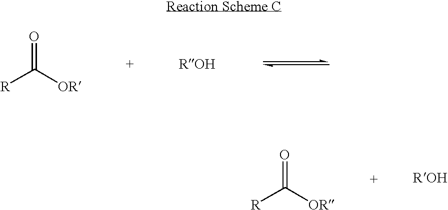

wherein an ester substrate (RC(O)OR′) and an alcohol substrate (R″OH) exchange “OR” groups to form an ester product (RC(O)OR″) and alcohol product (R′OH). Again, as with the prior reaction, the ester substrate and alcohol are part of an equilibrium reaction with the product ester and product alcohol.

Further embodiments of the process of the present invention are described with respect to the esterification reaction. However, a person skilled in the art would readily know how to apply this information to each of the reaction classes described above. For example, based on the teachings herein, a person skilled in the art would readily be able to select solid reactants, solvents, solid acid catalysts, process conditions, catalyst level, etc. for the individual reaction classes described above.

1. Non-Electrolyte Solutes

The non-electrolyte solute comprises a solute that is substantially non-ionized at the process conditions. For example, a substantially non-ionized solute is one wherein at least about 95% of the total solute molecules are non-ionized.

In one embodiment, the non-electrolyte solute product comprises an ester. Advantageously, the process of the present invention, as described above, can be used to prepare high boiling, low volatility esters in a relatively more efficient and/or productive manner. In one embodiment, for example, the process may be used to prepare intermediates useful in the preparation of imaging contrast agents such as those depicted in the following reaction scheme.

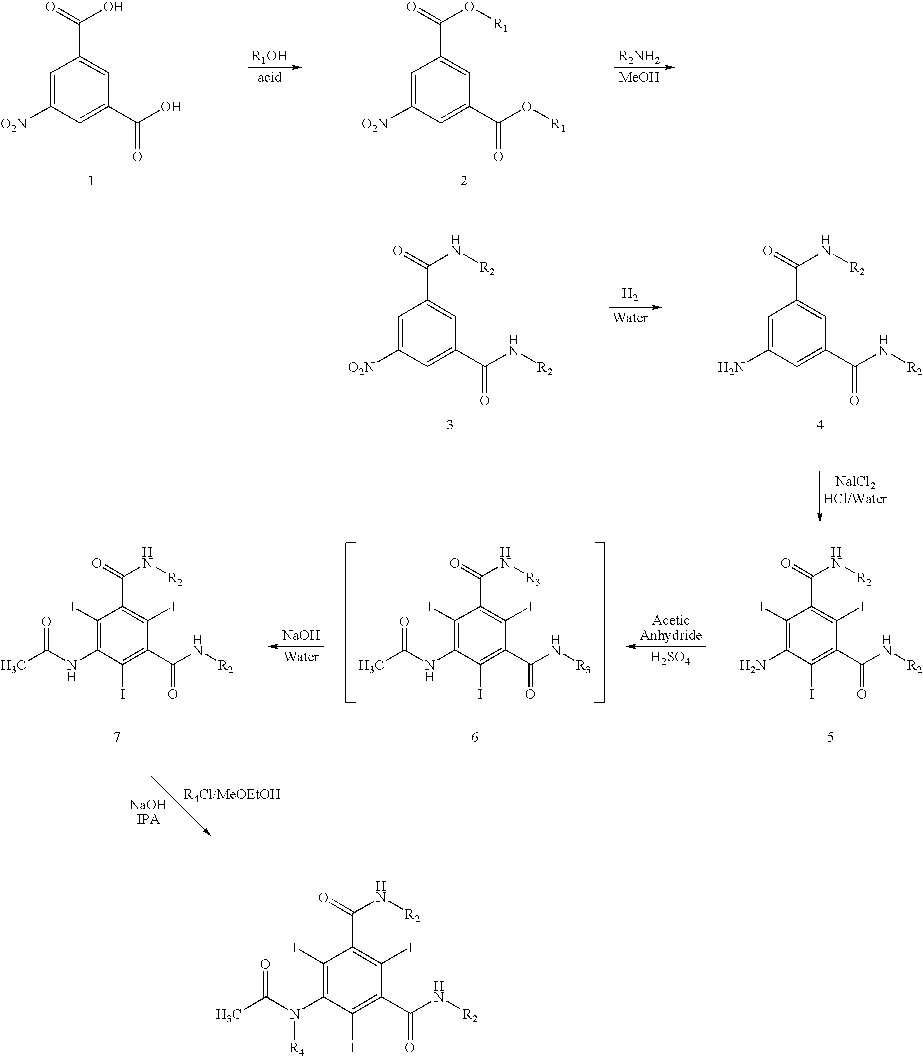

Esterification reactions may be catalyzed with an acid catalyst and, in the process of the present invention, a solid (or “supported”) acid catalyst is used. Advantageously, however, not all of the solid acid catalyst that needs to be used in the reaction is introduced to the reaction mixture at the outset. Instead, little or no solid acid catalyst is preferably included in the reaction mixture at the initiation of reaction, but then, after an appreciable conversion of carboxylic acid substrate to product ester, solid acid catalyst is added to the reaction mixture to drive the reaction to the desired degree of conversion.

2. Solid Reactants

The “R” group(s) of the substrate of Reaction Schemes A, B or C above, i.e. carboxylic acids (RCOOH), alcohols (ROH) and esters (RCOOR′), is/are selected such that the substrate is a solid at ambient conditions. In one embodiment, the “R” group(s) of the substrate of Reaction Schemes A, B or C above can be a substituted methyl, a substituted or unsubstituted linear, branched or cyclic alkyl, alkenyl, or alkynyl group having 2 to 30 carbon atoms in the main chain, a heteroaromatic or aromatic 5 or 6 membered cyclic structure, or a heteroaromatic or aromatic fused bicyclic structure; wherein the substituted methyl, alkyl, alkenyl, alkynyl, and cyclic structures are substituted with halo, nitro, cyano, amido, aryl, alkyl, alkoxy, aryloxy, hydroxyl or carboxy moieties.

In another embodiment, the solid reactant comprises a carboxylic acid substrate. The carboxylic acid substrate of the present invention produces a corresponding product ester that has one or more ester moieties and is relatively non-volatile (e.g., has a partial pressure less than about 75 mm Hg at the normal boiling point of the reacting alcohol). The carboxylic acid substrate can have one or more carboxylic acid moieties and the product ester can have one or more ester moieties. Provided the carboxylic acid is a solid at ambient conditions, both the carboxylic acid and ester can have a substituted methyl, substituted or unsubstituted linear, branched, or cyclic alkyl, alkenyl or alkynyl group having 2 to 30 carbon atoms in the main chain, a heteroaromatic or aromatic 5 or 6 membered cyclic structure, or a heteroaromatic or aromatic fused bicyclic structure wherein the substituted methyl, alkyl, alkenyl, alkynyl, and cyclic structures are substituted with halo, nitro, cyano, amido, aryl, alkyl, alkoxy, aryloxy, hydroxyl or carboxy moieties. Exemplary cyclic structures are phenyl, furyl, thienyl, pyridyl, oxazolyl, isoxazolyl, oxadiazolyl, pyrrolyl, pyrazolyl, triazolyl, tetrazolyl, imidazolyl, pyrazinyl, pyrimidyl, pyridazinyl, thiazolyl, thiadiazolyl, biphenyl, naphthyl, indolyl, isoindolyl, indazolyl, quinolinyl, isoquinolinyl, benzimidazolyl, benzotriazolyl, imidazopyridinyl, benzothiazolyl, benzothiadiazolyl, benzoxazolyl, benzoxadiazolyl, benzothienyl, benzofuryl and the like.

In one embodiment, the carboxylic acid substrate corresponds to an aromatic carboxylic acid compound of Formula 1 and the product ester corresponds to a compound of Formula 1A

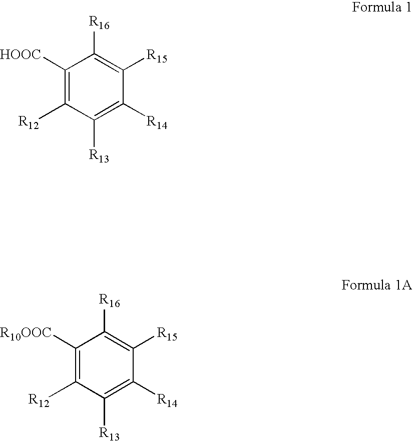

wherein

R₁₂, R₁₃, R₁₄, R₁₅ and R₁₆ are independently hydrogen, hydrocarbyl, substituted hydrocarbyl, halogen, heterocyclo, alkoxy, alkenoxy, alkynoxy, aryloxy, hydroxy, protected hydroxy, acyl, acyloxy, nitro, amido, cyano, thiol, hydroxycarbonyl or alkoxycarbonyl; and

R₁₀ is substituted or unsubstituted aryl or C₁-C₂₀ straight, branched or cyclic alkyl, alkenyl or alkynyl wherein the substituted aryl, alkyl, alkenyl or alkynyl groups are substituted with halo, alkyl, aryl, alkoxy, alkenoxy, alkynoxy, aryloxy, hydroxyl, nitro, cyano or amido.

Preferably, at least one of R₁₂, R₁₃, R₁₄, R₁₅ or R₁₆ is hydroxycarbonyl or alkoxycarbonyl. Alternatively, two or more of R₁₂, R₁₃, R₁₄, R₁₅ or R₁₆ is hydroxycarbonyl or alkoxycarbonyl and R₁₀ is a straight carbon chain having one to six carbon atoms, the carbon chain optionally substituted with alkoxy, aryloxy, aryl, cyano, nitro or halo.

In one preferred embodiment, the carboxylic acid substrate corresponds to a compound of Formula 2 and the product ester corresponds to a compound of Formula 2A

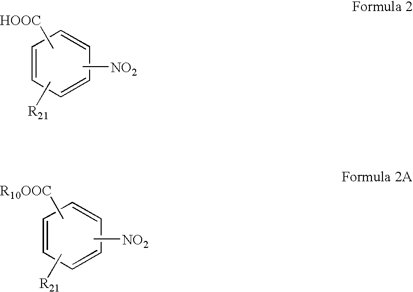

wherein

R₂₁ is hydrogen, —COOH, —COOR₁₁, hydrocarbyl, substituted hydrocarbyl, halogen, heterocyclo, alkoxy, alkenoxy, alkynoxy, aryloxy, hydroxy, protected hydroxy, acyl, acyloxy, nitro, amido, cyano, thiol, ketal, or acetal; R₁₁ is hydrogen, substituted or unsubstituted aryl or C₁-C₂₀ straight, branched or cyclic alkyl, alkenyl or alkynyl wherein the substituted aryl, alkyl, alkenyl or alkynyl groups are substituted with halo, alkoxy, alkenoxy, alkynoxy, aryloxy, hydroxyl, nitro, cyano or amido; and R₁₀ is defined as above. Preferably, R₂₁ is hydrogen, —COOH or —COOR₁₁.

In another preferred embodiment, the carboxylic acid substrate corresponds to a compound of Formula 3 and the product ester corresponds to a compound of Formula 3A

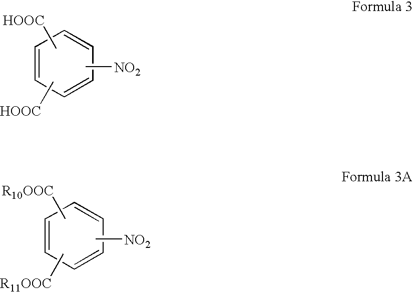

wherein R₁₁ and R₁₀ are defined as above.

In one particularly preferred embodiment in which the carboxylic acid substrate corresponds to Formula 3 and the product ester corresponds to Formula 3A, the nitro group is attached to C5 and the carboxylic acid or ester moieties are attached to C1 and C3; in this embodiment, the carboxylic acid substrate and the product ester are represented by Formulae 4 and 4A, respectively

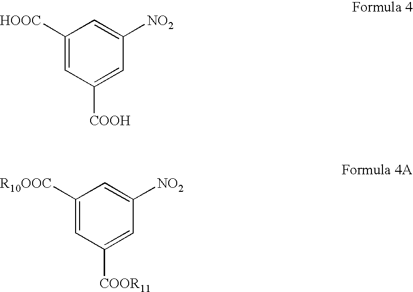

wherein R₁₀ and R₁₁ are defined as above. Preferably, R₁₀ is methyl, ethyl or propyl and R₁₁ is methyl, ethyl or propyl; more preferably, R₁₀ is methyl and R₁₁ is methyl.

In a further embodiment, a carboxylic acid substrate having more than one carboxylic group can form esters having different “R” groups attached to the C(O)O— moiety (e.g. a mixed ester). In order to facilitate mixed ester formation two or more alcohol substrates are added to the reaction mixture.

3. Solvent

In one embodiment, the solvent comprises an alcohol. In a further embodiment, the alcohol can be the reactant, R′OH or R″OH in esterification, etherification, transetherification or transesterification reactions depicted in Reaction Schemes A, B and C above. Typically, the alcohol (R′OH or R″OH) may be R₂₂OH wherein R₂₂ is substituted or unsubstituted aryl or C₁-C₂₀ straight, branched or cyclic alkyl, alkenyl or alkynyl wherein the substituted aryl, alkyl, alkenyl or alkynyl groups are substituted with halo, cyano, aryl, alkyl, alkoxy, alkenoxy, alkynoxy, aryloxy, hydroxyl, nitro, amido. Particularly for the etherification reaction, substituted and unsubstituted phenol and naphthol can be used. Preferred alcohols are in the liquid phase at 100° C., or have a favorable azeotrope with water so as to facilitate easy separation of by-product water from the reaction by distillation. Alcohols having a boiling point of less than 100° C. may be used; when such alcohols are used, however, the reaction vessel is preferably pressurized to maintain the alcohol in the liquid phase while maximizing the solubility of the product ester. In addition, the boiling point of the alcohol is preferably at least 40° C. less than the boiling point of the product ester.

Exemplary alcohols are straight, branched or cyclic C₁-C₁₂ alcohols. By way of further example, the alcohol can be a straight, branched or cyclic C₁-C₈ alcohol; particularly, 2-ethylhexyl alcohol. Presently preferred alcohols are straight or branched C₁-C₄ alcohols; particularly, methanol.

Reducing the volume of the alcohol is advantageous; especially, when a low boiling alcohol (normal boiling point <100° C.) such as methanol is the alcohol. Methanol volume reduction improves reactor payload and makes removing the water from the methanol easier.

In the process of the present invention, the alcohol also preferably serves as the solvent or, alternatively, is combined with one or more other miscible solvents to form a solvent system. In general, when an alcohol is combined with one or more other miscible solvents (e.g., a solvent system is formed), the amount of the alcohol in the solvent system can be from about a stoichiometric amount of alcohol needed for the conversion contemplated to an amount where substantially all of the solvent comprises the alcohol. The one or more other solvents miscible with the alcohol can be polar solvents such as ethyl acetate, dioxane, dimethyl sulfoxide, dimethyl formamide, dimethyl acetamide or similar solvent capable of solubilizing the solid reactant substrate and the product.

4. Solid Acid Catalyst

The catalyst for the process of the present invention is generally known as a solid acid. Typically, solid acid catalysts are porous materials with the reaction being catalyzed within the pores of the solid acid catalysts. In general, solid acid catalysts include strong cation exchange resins and strong acid resins. Advantageously, the solid acids are easily removed from the reaction mixture by filtration of the reaction mixture, where the mother liquor contains the product ester and the solid acid is separated from the mother liquor.

Preferably, the solid acid catalyst is a strong cation exchange resin, which comprises crosslinked, vinylaromatic polymer beads functionalized with strongly acidic moieties. The acidic moieties may be a strong acid group that has a pKₐ less than about 2. Commercially available strong cation exchange resins are functionalized with sulfonic acid groups or salts thereof. For the transformation of the present invention, the strong cation exchange resins are in the acid rather than the salt form. The crosslinked, vinylaromatic polymer beads can be partially or fully functionalized with strongly acidic moieties. In particular, the polymer beads can be surface functionalized with strongly acidic functional groups to a cation-exchange capacity of from about 0.1 to about 2.5 meq/g and an inner volume of unfunctionalized polymer. Alternatively, the cross linked, vinylaromatic polymer beads can be functionalized with strongly acidic functional groups to a cation-exchange capacity of at least about 2.5 meq/g; preferably, a cation-exchange capacity from about 3.0 meq/g to about 5.0 meq/g; particularly, a cation-exchange capacity from about 3.5 to about 4.5 meq/g. In addition, the crosslinked, vinylaromatic polymer beads can be functionalized with strongly acidic functional groups throughout the volume of the polymer bead.

Additionally, the strong cation exchange resins can be gel resins or macroreticular resins. Gel resins have a dense internal structure with no discrete pores and macroreticular resins have a porous, multichannelled structure. Strong cation exchange resins are available under the tradenames Amberlite®, Amberlyst®, Amberjet®, Dowex® and Duolite®. They are characterized by the minimum total exchange capacity and the water retention capacity in percent. An exemplary gel resin is Rohm & Haas IR-120. Presently preferred resins are macroreticular strong cation exchange resins; particularly those available under the tradenames Amberlite® and Amberlyst®. Exemplary resins are Dowex M-31 and Rohm & Haas FPC-22. These resins are sulfonic acid functionalized resins, with about 40% to about 60% water retention capacity and a total exchange capacity of about 1.5 meq/mL to about 2.3 meq/mL or about 3.0 meq/g to about 5.0 meq/g; preferably, about 1.6 meq/mL to about 2.0 meq/mL or about 35 meq/g to about 4.5 meq/g; more preferably, about 1.7 meq/mL to about 1.9 meq/mL or about 3.8 meq/g to about 4.2 meq/g.

Generally, the total amount of strong cation exchange resin used in the present process is not narrowly critical. The amount of the resin can be varied depending on the reaction (e.g. schemes A, B or C), and the desired conversion time and the temperature chosen for the reaction, and a person skilled in the art would readily be able to determine the total amount of solid acid catalyst to be used in the specific reactions of the invention described herein. Using the esterification reaction as an example, a total amount of solid acid catalyst used in the process is typically up to about 12 moles of carboxylic acid substrate per hours of reaction time per equivalents of solid acid catalyst. The values below are based on the dry weight of the solid acid catalyst. For example, the amount of the dry solid acid catalyst used to catalyze the esterification of the current process is from about 0.25 moles of carboxylic acid substrate per hours of reaction time per equivalents of solid acid catalyst (0.25 mol acid/hr/eq resin) to about 12 mol acid/hr/eq resin. The solid acid catalyst can be apportioned between a first step and a second step of the process as described below. Presently preferred ranges of the total amount of solid acid catalyst used in the first and second steps are from about 0.25 mol acid/hr/eq resin to about 6 mol acid/hr/eq resin; from about 0.5 mol acid/hr/eq resin to about 6 eq mol acid/hr/eq resin; from about 1.25 mol acid/hr/eq resin to about 6 mol acid/hr/eq resin; from about 0.25 mol acid/hr/eq resin to about 1.25 mol acid/hr/eq resin; from about 0.5 mol acid/hr/eq resin to about 1.25 mol acid/hr/eq resin; from about 0.25 mol acid/hr/eq resin to about 0.5 mol acid/hr/eq resin. One of ordinary skill in the art would select less catalyst to minimize the frequency of catalyst regeneration or replacement while lengthening the reaction time. If a specific reaction time was desired, for example, to avoid holding reaction mixture between stages, one of ordinary skill in the art would choose to use a greater amount of catalyst to minimize reaction time. In this case, preferred ranges of the total amount of solid acid catalyst used in the first and second steps are from about 0.5 mol acid/hr/eq resin to about 12 mol acid/hr/eq resin; from about 1.25 mol acid/hr/eq resin to about 12 mol acid/hr/eq resin; and from about 6 mol acid/hr/eq resin to about 12 mol acid/hr/eq resin.

### C. Esterification Reaction Process

a. Maximizing Conversion

Generally, the temperature of the process depends on the solubility of the product ester and the boiling point of the alcohol. Minimizing operating temperature is advantageous to extend resin life, and to lower process operating pressures. Depending on the particular reactants, lowering temperatures may also give less ether byproduct formation relative to the conversion of starting organic acid. For example, the product ester solubility increases with increasing temperature (e.g., dimethyl-5-nitro-isophthalate has a solubility of about 58 wt. % at 100° C. and a solubility of about 1 wt. % at 25° C.). Advantageously, the increasing solubility with increasing temperature decreases the amount of alcohol used in the reaction. Stated another way, the amount of alcohol needed to solubilize the product ester is not as great due to the increase in the solubility of the product ester in the alcohol at higher temperatures. Additionally, the maximum reaction temperature at atmospheric pressure depends on the boiling point of the alcohol. Accordingly, if the desired reaction temperature is greater than the boiling point of the alcohol at atmospheric pressure, the reaction can be carried out at a pressure higher than atmospheric pressure. When the product ester is dimethyl-5-nitro-isophthalate and the alcohol is methanol as in Example 1, presently preferred reaction temperature ranges are from about 100° C. to about 120° C., from about 100° C. to about 110° C.; and particularly, about 100° C.

Preparation of dimethyl-5-nitro-isophthalate (diester) solutions having a concentration of greater than 70 wt. % obtained by increase of the temperature to above 100° C. is surprising because the maximum solubility of the diester obtained below the normal boiling point of methanol is 7 wt. %.

It has been discovered that conversion of carboxylic acid substrate to ester can be maximized by converting a portion of the carboxylic acid substrate to the product ester prior to contact of the reaction mixture with the bulk of the solid acid catalyst. In one embodiment, the process of the present invention comprises a step-wise process. In a first step (sometimes referred to as a stage), a reaction mixture comprising the carboxylic acid substrate and alcohol are formed, optionally in the presence of solid acid catalyst, and a reaction between the two is initiated and proceeds until the molar ratio of the product ester to the carboxylic acid substrate in the reaction mixture is at least 1:3, 1:5, 1:10, 1:15, 1:20 or more, respectively. In the second step, solid acid catalyst is introduced to the reaction mixture and reaction between the carboxylic acid substrate and alcohol is allowed to proceed until the molar ratio of the product ester to the carboxylic acid substrate in reaction mixture is at least 9:1, respectively. In addition, proportionately more solid acid catalyst is preferably introduced in the second step than is present in the reaction mixture in the first step. Stated another way, at least 50%, 55%, 60%, 65%, 70%, 75%, 80%, 85%, 90%, 95% or more of the solid acid catalyst is added to the reaction mixture after at least 25%, 30%, 40%, 50%, 60%, 70%, 80%, 90% or more of the carboxylic acid substrate is converted to the product ester.

Referring again to Reaction Scheme A, in an esterification reaction the reactants and products reach an equilibrium. In order to favor the product ester, water is removed from the reaction mixture; the water removal provides a driving force to move the equilibrium position farther to the right to produce more of the product ester. The removal of water can be accomplished by methods known in the art such as distillation, pervaporation or use of a drying agent (e.g. magnesium sulfate or molecular sieves). Accordingly, water is removed during the reaction of a carboxylic acid substrate with an alcohol and can be removed once the reaction is substantially completed as described below.

Generally, the reaction comprising the first and second steps of the process may proceed for up to about 75 hours. The reaction time depends upon the temperature, amount of solid acid catalyst, the desired completeness of reaction, the starting composition for the reaction and the removal of water. In one embodiment, the temperature and amount of solid acid catalyst are selected to provide a reaction time of up to about 30 hours; further, a reaction time of up to about 15 hours; still further, a reaction time of up to about 10 hours.

A second step involving the reaction mixture preferably produces conversion of at least about 95% when the carboxylic acid substrate is measured on a molar basis. After the conversion of the substrate to product is at least about 95% complete, the resulting product ester solution is separated from the solid acid catalyst by methods known in the art (e.g. filtration). Depending on the product specifications desired, the product solution may be further dried after separation of the product mixture from the solid acid catalyst. The solution is easier to dry when it is no longer in contact with the solid acid catalyst.

One reason the product ester is easier to dry once it is not in contact with the solid acid catalyst is that it catalyzes a side reaction wherein two molecules of alcohol react to form a dialkyl ether and water. Therefore, contact of the product ester/alcohol mixture with the solid acid catalyst produces more water even if the reaction wherein the product ester is produced is substantially completed.

Water removal can be achieved by pervaporation, distillation, desiccation, addition of water scavenging agents and the like. Useful water scavenging agents are compounds that react with water including carboxylic acid anhydrides, carboxylic acid chlorides, oleum and the like. Additionally, water scavenging agents that absorb water are useful; for example, anhydrous inorganic salts that form hydrates (e.g., magnesium sulfate), molecular sieves and the like. A presently preferred method of water removal is contact of the product ester solution with a dry gas; particularly, contacting the product ester solution with alcohol vapor that has a water content of less than the water content of the vapor that is in equilibrium with the product ester solution. Preferably, the wt. % of water in the dry alcohol vapor is less than about 50% of the wt. % of water in the vapor that is in equilibrium with the product ester solution. More preferably, the wt. % of water in the dry alcohol vapor is less than about 10% of the wt. % of water in the vapor that is in equilibrium with the product ester solution.

Generally, adding dry alcohol vapor is equivalent to adding liquid alcohol and external heat. Adding liquid alcohol and an appropriate level of external heat provides alcohol vapor. Accordingly, when alcohol vapor is used, the alcohol vapor can be provided by alcohol liquid and external heat.

Additionally, the reaction mixture in both steps is heated and if necessary, the pressure is increased to increase the solubility of the product ester in the alcohol solvent and maintain the solvent in the liquid state. For example, a temperature of at least about 100° C. increases the solubility of dimethyl-5-nitro-isophthalate in methanol as described in more detail in Example 1. The temperature and pressure are set to solubilize the carboxylic acid and ester; having the product ester in solution facilitates the separation of the solid acid catalyst. In addition, the product ester solution can be used directly in a subsequent reaction step. After water is further removed post separation from the resin, the product ester solution is even more suitable for direct use in a subsequent reaction step leading to a contrast agent. The process of the present invention advantageously results in a product ester, such as those of Formulae 1A-4A, that contains less than about 3 wt. % water; preferably, less than about 2 or 1 wt. % water; more preferably, less than about 0.9, 0.8, 0.7, 0.6, 0.5, 0.4 or 0.3 wt. % water. For example, the present process results in a product ester of Formulae 1A-4A, which contains less than a measurable amount of sulfuric acid as compared to most commercial processes wherein sulfuric acid is used as an esterification catalyst. By way of further example, the product ester of Formulae 1A-4A contains less than about 3 wt. % of the starting acid of Formulae 1-4; preferably, less than about 2.5, 1.5 or 1 wt. % of the starting acid of Formulae 1-4; more preferably, less than about 0.9, 0.6, 0.4 or 0.2 wt. % of the starting acid of Formulae 1-4. In addition, the process preferably results in at least about 97% of the carboxylic acid substrate of Formulae 1-4 being converted to the product ester of Formulae 1A-4A.

Further, in one embodiment, the ester or ether product can be crystallized and separated from the reaction mixture. Generally, a higher conversion of substrate to product allows for more facile crystallization of the product. When the product forms crystals, the reaction mixture can be separated from the product crystals by appropriate means.

In one exemplary embodiment, when the diester reaction completion was greater than about 99.5%, the product diester solution formed large, needlelike crystals. The needlelike crystals had an interlacing three-dimensional structure, which resembled rolled fiberglass insulation in appearance. When the interlaced needlelike crystal structure described above formed, the mother liquor was drained without the aid of a filtering device or medium while the three dimensional crystal structure remained intact. Draining of the mother liquor removed 90% of the unreacted species and the purity of the remaining diester crystals was very high. The diester crystals could then be melted and used as a starting material in the downstream synthetic step.

b. Process Schematic

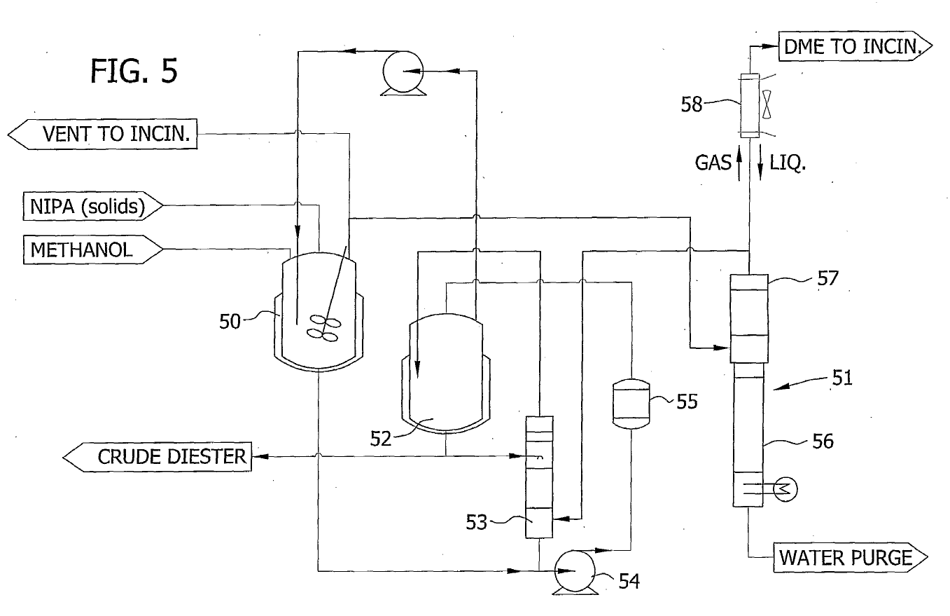

Referring to FIG. 5, an ester (e.g., dimethyl-5-nitrophalate (“diester” as in Example 7)) is prepared by esterifying a carboxylic acid substrate (e.g., NIPA) in an alcohol solvent (e.g., methanol). At start up of the process, a carboxylic acid substrate is introduced into the mix tank 50 which is then filled with nitrogen. A mix tank agitator is started and the methanol stream is then introduced into the carboxylic acid substrate. Heat is applied to the jacket. As the mix tank pressure increases, the nitrogen inert gas is vented through the mix tank vent to the incinerator so that the headspace of mix tank 50 is methanol vapor; when the headspace contains methanol vapor, the vent is closed. Heating continues until the desired operating pressure is reached. Afterwards, a short hold period (approximately 15 minutes) can be useful to line out the control of tank pressure. Methanol gas is diverted from the blower into mix tank 50 and returned to a methanol dryer distillation column 51. The methanol gas for mix tank 50 is typically supplied by the off-gas from a reactor 52, however, it may be taken either directly from the methanol column 51 or the exit of the water stripper column 53 depending on the cycle step of the reactor 52. Methanol gas continues to sparge and dry the contents of the reactor 52 for an extended time (circa 13 hrs). At the end of the sparge, about 60 to 80% of the starting carboxylic acid has been esterified. Heat is adjusted to the jacket of the mix tank 50 to maintain the mix tank 50 at a desired inventory (typically either constant weight or constant level) throughout the sparge. The contents of the mix tank 50 are then transferred into an empty reactor 52 as a solution. It is convenient to filter the solution on transfer or the solution can be continuously filtered as part of the pumparound loop during the reactor 52 cycle.

After the reactor 52 receives a new batch, the pumparound loop is started. The reaction mixture comprising carboxylic acid, methanol and ester is circulated by pump 54 to a resin bed 55. Methanol vapor from the methanol dryer column 51 is fed through the water stripper column 53 and exits the top of the column where it is sparged into reactor 52, and then vented from reactor 52 onto the system blower. The water stripper column 53 is not essential for the practice of this process, but provides added benefit in downsizing the amount of recycle methanol gas needed to run the process. The resin bed 55 contains a strong cation exchange resin. The water wet volume of resin should be consistent with the length of the reactor sparge cycle for the amount of carboxylic acid substrate. More or less resin could be used, depending on the desired length of the reactor sparge cycle. The pumparound loop and alcohol sparging is continued for the desired time (e.g., hours) at an appropriate flow rate. Methanol vapor flow and pumparound flowrates would require adjustment depending on the volume of resin used in the design, the size of the batch (amount of carboxylic acid substrate), and the concentration of the batch (amount of methanol versus ester/carboxylic acid). Heat is adjusted to the jacket of the reactor 52 to control the inventory (typically performed at constant weight or constant level) of the reactor 52 during the sparge.

When the sparge is complete, the pumparound loop is taken off-line and the batch is further dried by continuing the methanol sparging. The reactor 52 is sparged with methanol vapor directly from the methanol dryer column 51.

Optionally, the solvent methanol can be recovered and recycled. The recovery and recycle of the methanol and/or co-solvent reduces the cost of the process and eliminates a potential waste stream. A distillation column can be used to remove water from the methanol. In the distillation column, the methanol is separated from water, exits the distillation column and enters a knockback condenser wherein the hot methanol liquid falls back into the dried gas stream. As the dialkyl ether is lower boiling than methanol, it exits the top of the knockback condenser. Advantageously, the dried methanol vapor is recycled and used as a sparge gas to dry the product ester solution. Due to this recycle process, the methanol vapor is not completely condensed and reevaporated as in a conventional condenser, thus, the energy input required is only that required for methanol reflux. Further, the gas can contact the liquid in a countercurrent flow arrangement. For example, in the counter current arrangement, a drier gas contacts a wetter liquid.

Methanol recovery comprises the methanol dryer column 51 in the schematic of FIG. 5, though other technologies could be used to effect a similar separation. The recovery system separates methanol, water and ether (e.g., dimethyl ether (DME)) mixture into three separate components. The column has two separation sections. The lower section 56 strips methanol from water. A water rich stream exits the bottom of the column 51. The upper section 57 uses a methanol rich reflux to enrich and dry the methanol vapors. A dry methanol rich stream exits the top of the methanol dryer column 51 as a vapor and is fed to a knockback condenser 58. The knockback condenser 58 effects a separation of the ether, as well as makes reflux for the enriching section 57 of the column 51 (i.e., the ether rich stream that exits the knockback condenser). The reboiler load for the column 51 could be live steam injection or a heat exchanger. The energy usage for the column 51 is predominantly limited to that required to make reflux. The vapor circulation minimizes energy usage to avoid unnecessarily condensing and revaporizing the methanol vapors sparging the reactors 50 and 52.

It is noted that when the operating pressure of column 51 is increased so that the condensing point of the alcohol is at about Tₘᵢₙ to Tₘₐₓ, the higher condensing point allows the knockback condenser 58 to be air-cooled, thereby eliminating cooling water requirements for this system.

When the step-wise conversion of the carboxylic acid substrate to the product ester as described above is combined with alcohol recovery and recycle, the resultant product stream is of very high purity with no waste stream. In addition, due to the high purity of the product stream, crystallization of the product ester is not necessary before directing the product stream to the next reaction step leading to a contrast agent.

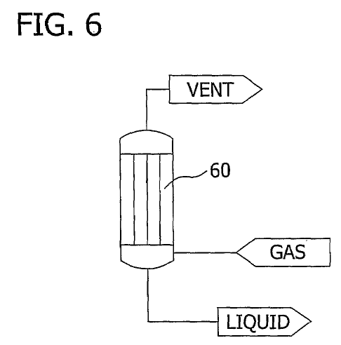

FIG. 6 represents a schematic diagram of a knockback condenser 60. The difference between a knockback condenser and a total or partial condenser is that the uncondensed vapor and the condensed liquid are at opposite ends of the equipment, precluding intimate contact and an equilibrium relationship for compositions of the gas and liquid streams. The knockback condenser is quite advantageous for this system, because of its high efficiency to separate dimethyl ether (vented in FIG. 6), which is a side reaction product, precluding the need for additional distillation equipment.

Alcohol recovery and recycle can further comprise recovery of the wet alcohol vapor by separation of a major portion of water and ether, to make a dry alcohol stream for recycle. The dry alcohol stream can be produced by the following process.

In another embodiment, the invention is directed to a separation process comprising separating alcohol, ether and water in a stream by feeding the stream to a distillation column system, the distillation column system comprising a lower stripping section, an upper enriching section and a knockback condenser. In this system, the feedstock is fed between the stripping and enriching sections, a water rich stream exits the bottom of the stripping section, an ether rich stream exits the knockback condenser and a product alcohol stream exits from between the top of the enriching section and the bottom of the knockback condenser. Preferably, the stream fed to the distillation column is a vapor and the alcohol stream is a vapor.

In a preferred embodiment, the alcohol is methanol and the ether is dimethyl ether. Further in this preferred embodiment, the stream fed to the distillation column system is a vapor containing methanol, dimethyl ether and water. In addition, the alcohol stream is a methanol vapor stream.

c. Process Alternatives

Generally, in a first embodiment, a carboxylic acid substrate forming a product ester having a partial pressure less than about 75 mm Hg at about the normal boiling point of the reacting alcohol is reacted with an alcohol in a step-wise conversion of a carboxylic acid substrate to a corresponding product ester wherein in a first step a reaction mixture comprising the carboxylic acid substrate and the alcohol produces conversion of at least about 25, 30, 35, 40, 45, 50, 55, 60, 65 or 70% of the carboxylic acid substrate measured on a molar basis, in a second step this reaction mixture is further reacted to produce conversion of at least about 95, 96, 97, 98 or 99% of the carboxylic acid substrate measured on a molar basis. In one embodiment, the reaction mixture contacts a solid acid catalyst wherein the ratio of the amount of the solid acid catalyst added in the second step to the amount of the solid acid catalyst added in the first step is greater than about 1:1. Preferably, the ratio of the amount of the solid acid catalyst added in the second step to the amount of the solid acid catalyst added in the first step is greater than about 2:1, 3:1, 4:1, 5:1, 6:1, 7:1, 8:1 or 9:1; more preferably, the ratio of the amount of the solid acid catalyst added in the second step to the amount of the solid acid catalyst added in the first step is greater than about 10:1, 15:1, 20:1, 30:1, 40:1, 50:1, 60:1, 70:1, 80:1 or 90:1; even more preferably, the ratio of the amount of the solid acid catalyst added in the second step to the amount of the solid acid catalyst added in the first step is greater than about 100:1, 200:1, 300:1, 400:1, 500:1, 600:1, 700:1, 800:1, or 900:1; even more preferably, the ratio of the amount of the solid acid catalyst added in the second step to the amount of the solid acid catalyst added in the first step is greater than about 1000:1.

In a second embodiment, a carboxylic acid substrate forming a product ester having a partial pressure less than about 75 mm Hg at about the normal boiling point of the reacting alcohol is reacted with an alcohol wherein the process comprises forming a reaction mixture by contacting a carboxylic acid substrate with an alcohol in the presence of a solid acid catalyst; drying the reaction mixture by contacting the reaction mixture with dry alcohol vapor wherein the dry alcohol vapor has a water concentration less than the water concentration of the vapor that is in equilibrium with the reaction mixture; removing a dialkyl ether byproduct from the dry alcohol vapor by refluxing the alcohol and venting the dialkyl ether byproduct, wherein the amount of dialkyl ether byproduct in the reaction mixture is less than about 0.3 wt. %. For example, the alcohol can be refluxed and the dialkyl ether byproduct removed from the system by using a knockback condenser as shown in FIG. 5.

In a third embodiment, the elements of a step-wise conversion of carboxylic acid to product ester and drying the reaction mixture are combined. Optionally, the process further comprises contacting a drier alcohol vapor stream with a wetter liquid reaction mixture stream in a countercurrent arrangement.

In many of the various embodiments described above, the preferred substrates, solvents, strong cation exchange resins, temperatures and pressures are as described above.

The following examples illustrate the invention.

### EXAMPLES

### Example 1

### Selection of Operating Temperature Using Diester Solubility Data and Ideal Solubility Theory

The solubility of dimethyl-5-nitrophthalate (“diester”) in methanol was determined through the normal boiling of methanol, and presented in the following table:

| | wt % |
|---|---|
| Temp C. | Diester |
| 25.5 | 1.0% |
| 31.1 | 1.2% |
| 34.1 | 1.4% |
| 34.6 | 1.5% |
| 39.6 | 1.9% |
| 44.0 | 2.3% |
| 44.3 | 2.3% |
| 52.4 | 3.5% |
| 54.1 | 3.7% |
| 61.2 | 5.7% |
| 65.3 | 7.0% |
| 65.4 | 7.0% |

Using glass pressure test tubes immersed in boiling water, an additional point for the solubility of diester at 100° C. was measured to be about 58 wt % diester.

Generally, the solubility was determined gravimetrically. A dilute slurry of diester and methanol was heated to a desired temperature. As needed, more diester solids were added to maintain a dilute slurry. The slurry was heated to a known temperature and stirred for approximately 15-30 minutes to assure the diester solids were equilibrated and the solution was saturated. Then, the stirring was stopped and the diester solids were allowed to settle. A sample of the supernatant liquid was obtained, the liquid was evaporated and the weight of the residual diester was measured. The weight of the residual diester solid was used to calculate the solubility of the diester solid in the methanol. This procedure was repeated for several different temperatures. Then, from the highest temperature, the procedure was repeated except the slurry was cooled to each sampling point and methanol was added instead of diester solids as necessary to maintain a dilute slurry. The difference in the solubility data sets between the heating data set and the cooling data set is an indication of how well equilibrium was obtained.

To interpret the solubility information, additional physical properties of diester were measured. The melting point was 124° C. (123-125° C.). The heat of fusion was 7422 cal/g·mole.

The solubility of diester, being a non-electrolyte, would obey the following thermodynamic relationship.

$$ln γ x = \frac{- Δ {H}^{F}}{R} ( \frac{1}{T} - \frac{1}{{T}_{m}} )$$

γ=activity coefficient

x=mole fraction

ΔH^{F}=Heat of Fusion

R=Gas Constant

T=Absolute Temperature

Tₘ=Melting Point Temperature (Absolute)

This relationship allows the weight % solubility versus temperature data to be mathematically transformed into an activity coefficient versus temperature data. The corresponding activity coefficient for each solubility data point is as follows:

| Temp C. | wt % Diester | Activ Coef |
|---|---|---|
| 25.5 | 1.0% | 36.3 |
| 31.1 | 1.2% | 33.7 |
| 34.1 | 1.4% | 32.4 |
| 34.6 | 1.5% | 32.1 |
| 39.6 | 1.9% | 30.1 |
| 44.0 | 2.3% | 28.4 |
| 44.3 | 2.3% | 28.3 |
| 52.4 | 3.5% | 25.3 |
| 54.1 | 3.7% | 24.7 |
| 61.2 | 5.7% | 21.9 |
| 65.3 | 7.0% | 20.4 |
| 65.4 | 7.0% | 20.5 |
| 100.0 | 58.0% | 3.5 |

Activity coefficients can be fitted to a Van Laar expression, where A and B are fitted parameters for the equation. The Van Laar expression is:

$$RT ln {γ}_{1} = {A ( 1 + \frac{A}{B} \frac{{x}_{1}}{{x}_{2}} )}^{- 2}$$

This equation can be rearranged to:

$$\frac{1}{\sqrt{RT ln {γ}_{1}}} = \frac{\sqrt{A}}{B} \frac{{x}_{1}}{{x}_{2}} + \frac{1}{\sqrt{A}}$$

The later equation allows straightforward application of linear regression. It is in the form Y=m×+b, with Y=1/√RT ln γ and X=x1/x2. The intercept, b, equals 1/√A. The slope, m, equals √A/B. Taking component 1 to be diester, and component 2 to be the solvent methanol, the Van Laar parameters for this solubility data are per the following table:

| | |
|---|---|
| A | 2146.89 |
| B | 761.6597 |

With the thermodynamic solubility equation and the Van Laar expression and fitted parameters, the entire solubility curve for saturated diester solutions can be calculated at any temperature through the melting point of diester. This solubility curve, along with the experimental data is shown in the graph in FIG. 1.

The analysis represented by the graph in FIG. 1 shows that temperatures slightly greater than 100° C. are preferred to maximize the solubility of diester in methanol.

Thus, if 100° C. is Tₘᵢₙ, Tₘₐₓ is calculated as (100° C.+0.8(124−100)) or 119° C., preferably (100° C.+0.6(124−100)) or 114° C., (100° C.+0.4(124−100)) or 110° C., or (100° C.+0.2(124−100)) or 105° C. Based on these calculations, the optimal temperature range for maximizing the solubility of diester was chosen as 101-105° C. in Example 2.

### Example 2

### Solid Acid Resin-Catalyzed Esterification

A 1 gallon autoclave contains a macro-reticular strong acid ion exchange resin (FPC-22). The initial charge of resin to the autoclave was 1070 ml of resin (water wet volume) or about 495 g of resin (net dry weight). The resin was used to catalyze three prior esterification reactions, without any regeneration cycles, prior to this experiment. 1170 g Nitro-iso-Phthalic Acid (NIPA) and 710 ml of Methanol was added to the autoclave. The mixture was heated to an operating temperature of 101-105° C. over one hour and then held an additional 25 minutes. After which, 81 ml/min of anhydrous methanol was fed to a vaporizer and then sparged into the reactor as methanol gas. The methanol gas was continuously removed from the reactor over 6 hours to maintain the reactor at a constant weight and in the desired temperature range of 101-105° C. Pressure in the reactor varied from 28 psig at the beginning of the methanol sparge to 34-36 psig at the end of the methanol sparge. The methanol gas from the reactor was condensed and analyzed for water by Karl Fischer. The water content of the condensed methanol varied from 6.8% wt/wt at the beginning of the methanol sparge to 0.4% wt/wt at the end of the sparge. The autoclave was unloaded through a sintered metal frit to separate the reaction mixture from the resin, which remained in the autoclave for subsequent experiments. The reaction mixture was allowed to slowly cool overnight to room temperature. The total product (liquid and solids) from the autoclave weighed 2.00 kg. Solids from the unloading pot analyzed 74% dry solids. The dry solids contained 0.29% NIPA, determined by base titration. The liquid from the unloading pot contained 3.4% dissolved solids. The dissolved solids analyzed 32% NIPA, also by base titration. The overall conversion of NIPA (MW=211) to diester (MW=239) was about 99.5% as illustrated in the summary material balance table:

Starting Amount 1170 g NIPA
Reaction Conversion 99.5% by Trial & Error
Liquors as % of Total Mass 11% by Trial & Error

| | | | | Total |
|---|---|---|---|---|
| Overall Balance | | Liquors | Solids | as NIPA |
| 6 | NIPA | 2 | 4 | 6 |
| 1318 | Diester | 5 | 1313 | 1164 |
| 676 | MeOH | 213 | 463 | |
| 2000 | Total | 220 | 1780 | 1170 |
| Analysis | % Solids | 3.4% | 74% | |
| | % NIPA in dry solids | 32% | 0.29% | |

### Example 3

### Comparative Isothermal Esterification with No Solid Acid Catalyst

In this example, the esterification was conducted at 120-125° C. without solid acid catalyst as compared to 101-105° C. with solid acid catalyst as in Example 2. The addition of solid acid catalyst as in Example 2 increases the conversion of the NIPA to diester. 1755 g Nitro-iso-Phthalic Acid (NIPA) and 925 ml of methanol was added to a 1 gallon autoclave. The mixture was heated to an operating temperature of 120-125° C. over two hours, and then held an additional hour. After which, 37 ml/min of anhydrous methanol was fed to a vaporizer, and then sparged into the reactor as methanol gas. The methanol gas was continuously removed from the reactor over 31 hours, to maintain the reactor at a constant weight and in the desired temperature range of 120-125° C. Pressure in the reactor varied from 52 psig at the beginning of the methanol sparge to 70-75 psig at the end of the methanol sparge. The methanol gas from the reactor was condensed and analyzed for water by Karl Fischer. The water content of the condensed methanol varied from 5.2% wt/wt at the beginning of the methanol sparge to 0.07% wt/wt at the end of the sparge. The autoclave was unloaded and the mixture allowed to slowly cool overnight to room temperature. The total product (liquid and solids) from the autoclave weighed 2.65 kg. Solids from the unloading pot analyzed 80% dry solids. The dry solids contained 6% NIPA, determined by base titration. For simplicity, any half ester of NIPA is included as part of the % NIPA value determined by the base titration. The liquid from the unloading pot contained 29% dissolved solids. The dissolved solids analyzed 46% NIPA, also by base titration. The overall conversion of NIPA (MW=211) to dimethyl-5-nitrophthalate (diester; MW=239) was about 91% as illustrated in the summary material balance table:

Starting Amount 1755 g NIPA
Reaction Conversion 91% by Trial & Error
Liquors as % of Total Mass 11% by Trial & Error

| | | | | Total |
|---|---|---|---|---|
| Overall Balance | | Liquors | Solids | as NIPA |
| 153 | NIPA | 40 | 113 | 153 |
| 1815 | Diester | 47 | 1768 | 1602 |
| 682 | MeOH | 212 | 470 | |
| 2650 | Total | 299 | 2351 | 1755 |
| Analysis | % Solids | 29% | 80% | |
| | % NIPA in dry solids | 46% | 6% | |

### Example 4

### Comparative Isobaric Esterification with No Solid Acid Catalyst

In this example, the esterification was conducted at an operating pressure of 40-42 PSIG without solid acid catalyst as compared to an operating temperature of 101-105° C. with solid acid catalyst as in Example 2. The addition of solid acid catalyst as in Example 2 increases the conversion of NIPA to diester. 3540 g Nitro-iso-Phthalic Acid (NIPA) and 2210 g of methanol was added to a 2 gallon reactor. The mixture was heated to an operating pressure of 40-42 PSIG over 40 minutes, and then held an additional 35 minutes. After which, 1.0 gallon per hour of anhydrous methanol was fed to a vaporizer, and then sparged into the reactor as methanol gas. The methanol gas was continuously removed from the reactor over 770 minutes, to maintain the reactor at a constant weight and in the desired pressure range of 40-42 PSIG. Temperature in the reactor varied from, 113° C. at the beginning of the methanol sparge to 110° C. at the end of the methanol sparge. The methanol gas from the reactor was condensed and analyzed for water by Karl Fischer. The water content of the condensed methanol varied from 2.2% wt/wt at the beginning of the methanol sparge to 0.29% wt/wt at the end of the sparge. The reactor was unloaded and the mixture allowed to slowly cool overnight to room temperature. The total product (liquid and solids) from the reactor weighed 6.05 kg. Solids from the unloading pot analyzed 68% dry solids. The dry solids contained 27% NIPA, determined by base titration. The liquid from the unloading pot contained 54% dissolved solids. The dissolved solids analyzed 52% NIPA, also by base titration. The overall conversion of NIPA (MW=211) to diester (MW=239) was about 63% as illustrated in the summary material balance table:

Starting Amount 3540 g NIPA
Reaction Conversion 63% by Trial & Error
Liquors as % of Total Mass 33% by Trial & Error

| | | | | Total |
|---|---|---|---|---|
| Overall Balance | | Liquors | Solids | as NIPA |
| 1319 | NIPA | 556 | 747 | 1303 |
| 2516 | Diester | 513 | 2020 | 2237 |
| 2215 | MeOH | 911 | 1302 | |
| 6050 | Total | 1980 | 4070 | 3540 |
| Analysis | % Solids | 54% | 68% | |
| | % NIPA in dry solids | 52% | 27% | |

### Example 5

### Solvent Drying Following Separation of Reaction Mixture from Solid Acid Catalyst

The invention is exemplified as in example 2, except that the reaction mixture was sparged with anhydrous solvent after separation of the solid acid catalyst. A 1 gallon autoclave contains a macro-reticular strong acid ion exchange resin (FPC-22). The initial charge of resin to the autoclave was 1070 ml of resin (water wet volume) or about 495 g of resin (net dry weight). The resin has been used to catalyze six prior esterification reactions, without any regeneration cycles, prior to the upcoming experiment. 1170 g Nitro-iso-Phthalic Acid (NIPA) and 710 ml of Methanol was added to the autoclave. The mixture was heated to an operating temperature of 101-105° C. over two hours, and then held an additional 30 minutes. After which, 91 ml/min of anhydrous methanol was fed to a vaporizer and then sparged into the reactor as methanol gas. The methanol gas was continuously removed from the reactor over 6 hours to maintain the reactor at a constant weight and in the desired temperature range of 101-105° C. Pressure in the reactor varied from 30 psig at the beginning of the methanol sparge to 34-35 psig at the end of the methanol sparge. The methanol gas from the reactor was condensed and analyzed for water by Karl Fischer. The water content of the condensed methanol varied from 6.6% wt/wt at the beginning of the methanol sparge to 0.4% wt/wt at the end of the sparge. The water content of the condensed methanol was substantially not changing with time for approximately one hour, as illustrated by the following measurements:

| Elapsed Time - Hrs | % Water in Condensed Methanol |
|---|---|
| 4 | 0.47 |
| 5 | 0.39 |
| 6 | 0.42 |

The autoclave was unloaded through a sintered metal frit to separate the reaction mixture from the resin, which remained in the autoclave for subsequent experiments. The reaction mixture was then sparged with anhydrous methanol gas at the same rate as previous for an additional 25 minutes after separation from the resin. The water content of the condensed methanol rapidly decreased with time, as illustrated by the following measurements:

| Elapsed Time - Minutes | % Water in Condensed Methanol |
|---|---|
| 2 | 0.23 |
| 12 | 0.13 |
| 22 | 0.08 |

After the second sparge was complete, the mixture was allowed to slowly cool overnight to room temperature. The total product (liquid and solids) from the autoclave weighed 2.05 kg. Solids from the unloading pot analyzed 92.6% Dry Solids. The dry solids contained 0.108% NIPA, determined by base titration. The liquid from the unloading pot contained 1.56% dissolved solids. The dissolved solids analyzed 23.4% NIPA, also by base titration. The overall conversion of NIPA (MW=211) to diester (MW=239) was about 99.7% as illustrated in the summary material balance table:

Starting Amount 1170 g NIPA
Reaction Conversion 99.7% by Trial & Error
Liquors as % of Total Mass 31% by Trial & Error

| | | | | Total |
|---|---|---|---|---|
| | Overall Balance | Liquors | Solids | as NIPA |
| 4.0 | NIPA | 2.3 | 1.4 | 3.7 |
| 1321 | Diester | 8 | 1314 | 1166 |
| 725 | MeOH | 620 | 105 | |
| 2050 | Total | 630 | 1420 | 1170 |
| Analysis | % Solids | 1.56% | 92.6% | |
| | % NIPA in dry solids | 23.4% | 0.108% | |

### Example 6

### Condenser Simulation to Compare Partial Condenser and Knockback Condenser

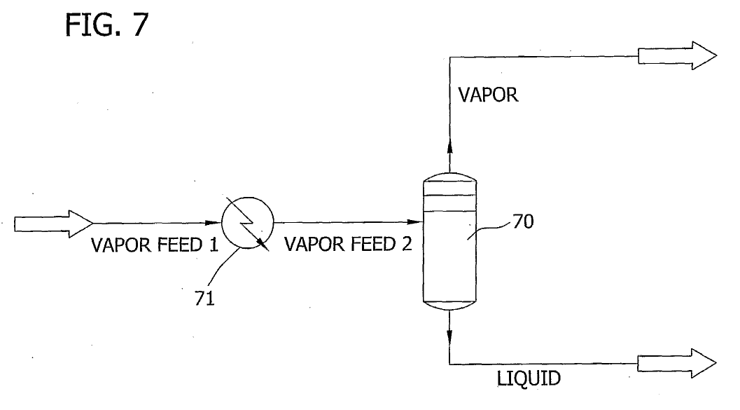

The schematic in FIG. 7 and stream information below simulates what happens in a partial condenser 70 that could be used in a distillation column. The vapor feed 1 (stream 72) is cooled by a heat exchanger 71 (vapor feed 2; stream 73) to condense a liquid where some vapor remains uncondensed. In a partial condenser 71, the exit liquid (stream 75) and vapor (stream 74) streams are in equilibrium with each other.

The amount of separation of dimethyl ether (DME) for this partial condenser is 0.1313/0.8000=16% of the inlet dimethyl ether. A total condenser would condense all the dimethyl ether resulting in no separation. A process of low or no separation would require additional distillation equipment to separate dimethyl ether from the methanol.

| Stream No. | 72 | 73 | 74 | 75 |
|---|---|---|---|---|
| Temp F. | 197.2292 | 182.2508 | 182.2508 | 182.2508 |
| Pres psia | 40.0000* | 40.0000 | 40.0000 | 40.0000 |
| Enth MMBtu/h | −0.086674 | −0.10115 | −0.00088892 | −0.10026 |
| Vapor mole fraction | 1.0000 | 0.010500 | 1.0000 | 0.00000 |
| Total lbmol/h | 1.0104 | 1.0104 | 0.0106 | 0.9998 |
| Total lb/h | 32.6177 | 32.6177 | 0.3799 | 32.2378 |
| Total std L ft 3/hr | 0.6558 | 0.6558 | 0.0081 | 0.6477 |
| Total std V scfh | 383.41 | 383.41 | 4.03 | 379.39 |

Flowrates in lb/h

| | | | | |
|---|---|---|---|---|
| Dimethyl Ether | 0.8000 | 0.8000 | 0.1313 | 0.6687 |
| Methanol | 31.8177 | 31.8177 | 0.2486 | 31.5691 |

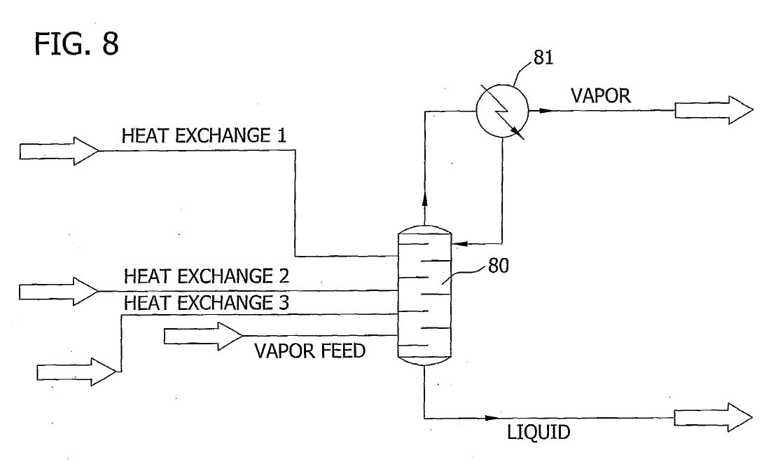

The schematic in FIG. 8 represents a knockback condenser 80. In knockback condenser 80, hot vapor feed (stream 87) enters the bottom of the condenser and hot liquid (stream 85) exits the bottom of the condenser. As the hot vapor rises in the condenser tube, heat is removed from the gas to effect condensation with the liquid falling down to hotter parts of the heat exchanger. Some uncondensed vapor (stream 86) exits the top of the condenser. In this simulation, heat is removed in streams 1, 2 and 3, (heat exchange 1 represents stream 82; heat exchange 2 represents stream 83; heat exchange 3 represents stream 84) which have no mass but only enthalpy (heat) content. The unit operation for this knockback condenser 80 is a distillation column. In this case, the column has a partial condenser in addition to the three side enthalpy inputs.

| Stream No. | 82 | 83 | 84 |
|---|---|---|---|
| Temp F. | 0.0000 | 0.0000 | 0.0000 |
| Pres psia | 0.0000* | 0.0000* | 0.0000* |
| Enth MMBtu/h | −0.0046015 | −0.0046015 | −0.0046015 |
| Vapor mole fraction | 0.00000 | 0.00000 | 0.00000 |
| Total lbmol/h | 0.0000 | 0.0000 | 0.0000 |
| Total lb/h | 0.0000 | 0.0000 | 0.0000 |
| Total std L ft 3/hr | 0.0000 | 0.0000 | 0.0000 |
| Total std V scfh | 0.00 | 0.00 | 0.00 |

Flowrates in lb/h

| | | | |
|---|---|---|---|
| Dimethyl Ether | 0.0000 | 0.0000 | 0.0000 |
| Methanol | 0.0000 | 0.0000 | 0.0000 |

| Stream No. | 85 | 86 | 87 |
|---|---|---|---|
| Temp F. | 197.2261 | 150.0000 | 197.2275 |
| Pres psia | 40.0000 | 40.0000 | 40.0000 |
| Enth MMBtu/h | −0.098582 | −0.0021176 | −0.086674 |
| Vapor mole fraction | 0.00000 | 1.0000 | 1.0000 |
| Total lbmol/h | 0.9843 | 0.0261 | 1.0104 |
| Total lb/h | 31.5485 | 1.0692 | 32.6177 |
| Total std L ft 3/hr | 0.6315 | 0.0243 | 0.6558 |
| Total std V scfh | 373.51 | 9.91 | 383.41 |

Flowrates in lb/h

| | | | |
|---|---|---|---|
| Dimethyl Ether | 0.0357 | 0.7643 | 0.8000 |
| Methanol | 31.5129 | 0.3048 | 31.8177 |

The total separation efficiency for dimethyl ether in this simulation is 0.7643/0.8000=95%. The knockback condenser 80 allows an easy separation of the ether from the alcohol. The vapor feed streams to the partial condenser 70 of FIG. 7 and the knockback condenser 80 are identical.

### Example 7

The equipment as shown in FIG. 5 was used to demonstrate the esterification process of the invention to make dimethyl-5-nitrophalate (diester) as follows. 2561 lbs of NIPA is charged to the mix tank 50. Mix tank 50 is then filled with nitrogen. A mix tank agitator is started and 1583 lbs of methanol liquid is added. Heat is applied to the jacket. As the mix tank pressure increases, the nitrogen inert gas is vented through the mix tank vent to the incinerator so that the headspace of mix tank 50 is methanol vapor; when the headspace contains methanol vapor, the vent is closed. Heating continues until an operating pressure of 40 psig is reached. After 40 psig is reached, a short hold period (approximately 15 minutes) can be useful to line out the control of tank pressure. Methanol gas is diverted from the blower into mix tank 50 and returned to the methanol dryer column 51. Methanol gas continues to sparge and dry the contents of the diester reactor 52 for an extended time (circa 13 hrs). At the end of the sparge, about 60 to 80% of the starting NIPA has been esterified. Heat is adjusted to the jacket of the mix tank 50 to maintain the mix tank 50 at a desired inventory (typically either constant weight or constant level) throughout the sparge. The contents of the mix tank 50 are then transferred into an empty diester reactor 52 as a solution. The pumparound loop is then started. NIPA/diester solution is pumped to a water stripper column 53 and through a resin bed 55. Methanol gas from the methanol dryer column 51 is fed through the water stripper column, sparged into diester reactor 52, and then onto the system blower. 328 liters (water wet volume) of resin would be consistent with a 14 hour reactor sparge cycle for the amount of NIPA used in this example. More or less resin could be used, depending on the desired length of the reactor sparge cycle. The pumparound loop and methanol sparging is continued for about 14 hours with a methanol vapor flow of 2200 lb/hr and a flow rate in the pumparound loop of 6000 lb/hr. Methanol vapor flow and pumparound flowrates would require adjustment depending on the volume of resin used in the design, the size of the batch (amount of NIPA), and the concentration of the batch (amount of MeOH versus diester/NIPA). Heat is adjusted to the jacket of the diester reactor 52 to control the inventory (typically performed at constant weight or constant level) of the diester reactor 52 during the sparge.

When the sparge is complete, the pumparound loop is taken off-line and the batch is further dried by continuing the methanol sparging by sparging the diester reactor 52 with methanol vapor directly from the methanol dryer column 51 distillation column. In order to operate the column 51 at 99-103° C., the operating pressure is 35-40 psig which increases the condensing point for methanol from 65° C. (1 atm). The higher condensing point allows the knockback condenser 58 to be air-cooled, thereby eliminating cooling water requirements for this system.

As various changes could be made in the above methods without departing from the scope of the invention, it is intended that all matter contained in the above description or shown in the accompanying drawings shall be interpreted as illustrative and not in a limiting sense.

1-43. (canceled)

44. A process having an operating temperature range, Tₘᵢₙ to Tₘₐₓ, at which solubility of a non-electrolyte solute in a solvent is maximized, the process comprising:
forming a mixture comprising the non-electrolyte solute and the solvent, wherein the solubility of the solute in the solvent is less than the ideal solubility at temperature, T, the temperature of the mixture being between Tₘᵢₙ and Tₘₐₓ; wherein
γₛₒₗᵤₜₑ is the activity coefficient of the solute in a two component mixture of the solute in the solvent at a temperature, T, derived from equation (5);
$$ln {γ}_{solute} {x}_{solute} = \frac{- Δ {H}_{solute}^{F}}{R} ( \frac{1}{T} - \frac{1}{{T}_{m ( solute )}} ) \qquad ( 5 )$$
γₛₒₗᵤₜₑ is the mole fraction of dissolved solute in a saturated two component mixture of solute and solvent at a temperature, T, in K;
R is the ideal gas constant in cal/K·mol;
−ΔH^{F}ₛₒₗᵤₜₑ is the heat of fusion in cal/g·mol of the solute;
Tₘ₍ₛₒₗᵤₜₑ₎ is the absolute melting temperature of the solute, in K;
Tₘᵢₙ is a temperature, in K, where an absolute value of γₛₒₗᵤₜₑ is greater than one and less than about 10 and the second derivative of a function of γₛₒₗᵤₜₑ with respect to temperature becomes a positive value; and
Tₘₐₓ is a temperature, in K, which is greater than Tₘᵢₙ and less than (Tₘᵢₙ+0.8(Tₘ₍ₛₒₗᵤₜₑ₎−Tₘᵢₙ)).

45. A process having an operating temperature range, Tₘᵢₙ to Tₘₐₓ, at which solubility of a non-electrolyte solute in a solvent is maximized, the process comprising forming a mixture comprising the non-electrolyte solute and the solvent, wherein the solubility of the solute in the solvent is less than the ideal solubility at a temperature, T, the temperature of the mixture being between Tₘᵢₙ and Tₘₐₓ; wherein
xₛₒₗᵤₜₑ is the mole fraction of dissolved solute in a saturated two component mixture of solute and solvent at a temperature, T;
Tₘ₍ₛₒₗᵤₜₑ₎ is the normal melting temperature of the solute;
Tₘᵢₙ is a temperature where the second derivative of a function of xₛₒₗᵤₜₑ with respect to temperature becomes a negative value; and
Tₘₐₓ is a temperature greater than Tₘᵢₙ and less than (Tₘᵢₙ+0.8(Tₘ₍ₛₒₗᵤₜₑ₎−Tₘᵢₙ)).

46. A process having an operating temperature range, Tₘᵢₙ to Tₘₐₓ, at which solubility of a non-electrolyte solute in a solvent is maximized, the process comprising forming a mixture comprising the non-electrolyte solute and the solvent, wherein the solubility of the solute in the solvent is less than the ideal solubility at a temperature, T, the temperature of the mixture being between Tₘᵢₙ and Tₘₐₓ; wherein
wt. %ₛₒₗᵤₜₑ is the wt. % of dissolved solute in a saturated two component mixture of solute and solvent at a temperature, T;
Tₘ₍ₛₒₗᵤₜₑ₎ is the normal melting temperature of the solute;
Tₘᵢₙ is a temperature where the second derivative of a function of wt. %ₛₒₗᵤₜₑ with respect to temperature changes becomes a negative value; and
Tₘₐₓ is a temperature greater than Tₘᵢₙ and less than about (Tₘᵢₙ+0.8(Tₘ₍ₛₒₗᵤₜₑ₎−Tₘᵢₙ)).

47. The process of claim 44 wherein the non-electrolyte solute comprises an ester or an ether.

48. The process of claim 47 wherein the ester is selected from compounds having structures corresponding to
(a) Formula 1A, 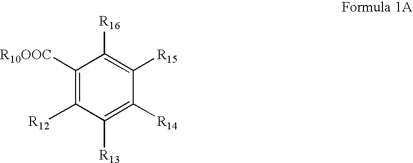
(b) Formula 2A, 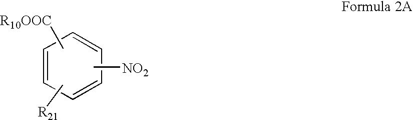
(c) Formula 3A, or 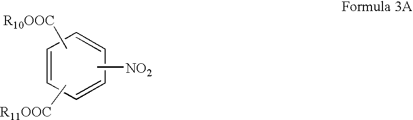
(d) Formula 4A 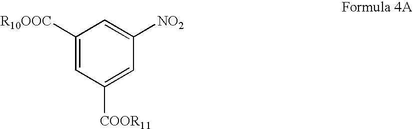
wherein
R₁₂, R₁₃, R₁₄, R₁₅ and R₁₆ are independently hydrogen, hydrocarbyl, substituted hydrocarbyl, halogen, heterocyclo, alkoxy, alkenoxy, alkynoxy, aryloxy, hydroxy, protected hydroxy, acyl, acyloxy, nitro, amido, cyano, thiol, hydroxycarbonyl or alkoxycarbonyl; and
R₁₀ is substituted or unsubstituted aryl or substituted or unsubstituted C₁-C₂₀ straight, branched or cyclic alkyl, alkenyl or alkynyl;
R₂₁ is hydrogen, —COOH, —COOR₁₁, hydrocarbyl, substituted hydrocarbyl, halogen, heterocyclo, alkoxy, alkenoxy, alkynoxy, aryloxy, hydroxy, protected hydroxy, acyl, acyloxy, nitro, amido, cyano, thiol, ketal, or acetal; and
R₁₁ is hydrogen or substituted or unsubstituted aryl or C₁-C₂₀ straight, branched or cyclic alkyl, alkenyl or alkynyl.

49. The process of claim 44 wherein Tₘₐₓ is less than about (Tₘᵢₙ+0.6(Tₘ₍ₛₒₗᵤₜₑ₎−Tₘᵢₙ)).

50. The process of claim 49 wherein Tₘₐₓ is less than about (Tₘᵢₙ+0.4(Tₘ₍ₛₒₗᵤₜₑ₎−Tₘᵢₙ)).

51. The process of claim 50 wherein Tₘₐₓ is less than about (Tₘᵢₙ+0.2(Tₘ₍ₛₒₗᵤₜₑ₎−Tₘᵢₙ)).

52. A process for preparing an ester from a carboxylic acid comprising
(a) reacting a carboxylic acid substrate with an alcohol to form a reaction mixture comprising the carboxylic acid substrate, the alcohol, and the ester wherein at least about 25% of the carboxylic acid substrate measured on a molar basis is converted to the ester;
(b) adding solid acid catalyst and contacting the reaction mixture with the solid acid catalyst to form a reaction product comprising the ester wherein at least about 97% of the carboxylic acid substrate measured on a molar basis is converted to ester.

53. The process of claim 52 further comprising
(c) crystallizing the reaction product and separating the crystallized reaction product from the reaction mixture.

54. The process of claim 52 wherein solid acid catalyst is added at step (a) and the ratio of the amount of the solid acid catalyst added in step (b) to the amount of the solid acid catalyst added in step (a) is greater than about 1:1.

55. The process of claim 54 wherein solid acid catalyst is added at step (a) and the ratio of the amount of the solid acid catalyst added in step (b) to the amount of the solid acid catalyst added in step (a) is greater than about 10:1.

56. The process of claim 52 wherein the carboxylic acid substrate and corresponding product ester are selected from compounds having structures corresponding to
(a) Formula 1 and 1A, respectively, 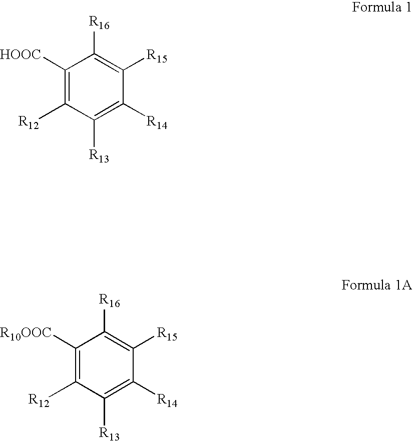
(b) Formula 2 and 2A, respectively, 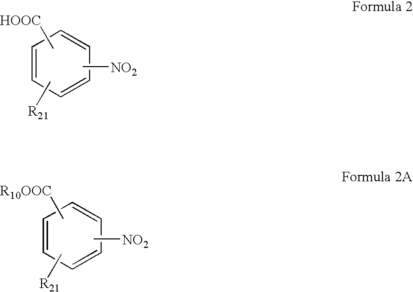
(c) Formula 3 and 3A, respectively, or 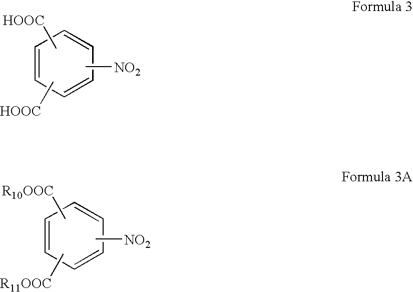
(d) Formula 4 and 4A, respectively 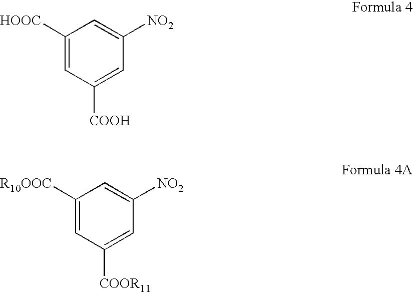
wherein
R₁₂, R₁₃, R₁₄, R₁₅ and R₁₆ are independently hydrogen, hydrocarbyl, substituted hydrocarbyl, halogen, heterocyclo, alkoxy, alkenoxy, alkynoxy, aryloxy, hydroxy, protected hydroxy, acyl, acyloxy, nitro, amino, amido, cyano, thiol, hydroxycarbonyl or alkoxycarbonyl; and
R₁₀ is aryl or substituted or unsubstituted C₁-C₂₀ straight, branched or cyclic alkyl, alkenyl or alkynyl;
R₂₁ is hydrogen, —COOH, —COOR₁₁, hydrocarbyl, substituted hydrocarbyl, halogen, heterocyclo, alkoxy, alkenoxy, alkynoxy, aryloxy, hydroxy, protected hydroxy, acyl, acyloxy, nitro, amido, cyano, thiol, ketal, or acetal; and
R₁₁ is substituted or unsubstituted aryl or C₁-C₂₀ straight, branched or cyclic alkyl, alkenyl or alkynyl.

57. The process of claim 52 wherein the alcohol has the formula of R₂₂OH wherein R₂₂ is C₁-C₂₀ straight, branched or cyclic alkyl.

58. The process of claim 52 wherein the solid acid catalyst is a strong acid cation exchange resin.

59. The process of claim 52 wherein the conversion is carried out at a temperature from about Tₘᵢₙ to about Tₘₐₓ;
wherein Tₘᵢₙ is a temperature, in K, where an absolute value of γₛₒₗᵤₜₑ is greater than one and less than about 10 and the second derivative of a function of γₛₒₗᵤₜₑ with respect to temperature becomes a positive value; and
Tₘₐₓ is a temperature, in K, which is greater than Tₘᵢₙ and less than (Tₘᵢₙ+0.8(Tₘ₍ₛₒₗᵤₜₑ₎−Tₘᵢₙ));
wherein γₛₒₗᵤₜₑ is the activity coefficient of the solute in a two component mixture of the solute in the solvent at a temperature, T, derived from equation (5);
$$ln {γ}_{solute} {x}_{solute} = \frac{- Δ {H}_{solute}^{F}}{R} ( \frac{1}{T} - \frac{1}{{T}_{m ( solute )}} ) \qquad ( 5 )$$
xₛₒₗᵤₜₑ is the mole fraction of dissolved solute in a saturated two component mixture of solute and solvent at a temperature, T, in K;
R is the ideal gas constant in cal/K·mol;
−ΔH^{F}ₛₒₗᵤₜₑ is the heat of fusion in cal/g·mol of the solute; and Tₘ₍ₛₒₗᵤₜₑ₎ is the absolute melting temperature of the solute, in K.

60. The process of claim 52 further comprising
drying the reaction mixture by contacting the reaction mixture with dry alcohol vapor wherein the water content (in wt. %) of dry alcohol vapor is less than the water content of the vapor (in wt. %) in equilibrium with the reaction mixture; and
feeding the dry alcohol vapor to a knockback condenser to separate a dialkyl ether byproduct from the alcohol, wherein the amount of the dialkyl ether byproduct in the reaction mixture is less than about 0.3 wt. %.

61. The process of claim 60 wherein the reaction mixture is dried by contacting it with the dry alcohol vapor via countercurrent flow.

62. The process of claim 60 wherein the water content (in wt. %) of dry alcohol vapor is about 50% less than the water content of the vapor (in wt. %) in equilibrium with the reaction mixture.

63. The process of claim 60 wherein the reaction mixture is removed from contact with the solid acid catalyst.

64. A process for separating a mixture of an alcohol, an ether and water, the process comprising:
introducing an aqueous mixture of an alcohol and an ether into a distillation column;
separating the mixture to produce water and a vapor comprising the ether and the alcohol;
introducing the vapor exiting an upper portion of the column into a lower portion of a knockback condenser; and
condensing the vapor in the knockback condenser to form an ether-rich vapor exiting an upper portion of the knockback condenser and an alcohol-rich liquid exiting the lower portion of the knockback condenser, wherein the ether-rich vapor contains at least about 50 wt. % of the ether in the mixture.

65. The process of claim 64 wherein the ether-rich vapor contains at least about 60 wt. % of the ether in the mixture.

66. The process of claim 64 wherein the alcohol-rich liquid is recycled to the upper portion of the column.

67. The process of claim 64 wherein the alcohol is methanol.

68. The process of claim 64 wherein the ether is dimethyl ether.
